SEQUENCE LISTING (85 sequences)

SEQ ID NO: 1 - protein, 300 aa, Homo sapiens
  REGION 1..300: note = misc_feature - Human DcR3 amino acid sequence
MRALEGPGLSLLCLVLALPALLPVPAVRGVAETPTYPWRDAETGERLVCAQCPPGTFVQRPCRRDSPTTCGPCPPRHYTQFWNYLERCRYCNVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLKLRRRLTELLGAQDGALLVRLLQALRVARMPGLERSVRERFLPVH

SEQ ID NO: 2 - protein, 5 aa, synthetic construct
  REGION 1..5: note = Synthetic: Heavy Chain (HC) CDR1 antibody F19
GYNWH

SEQ ID NO: 3 - protein, 16 aa, synthetic construct
  REGION 1..16: note = Synthetic: HC CDR2 antibody F19
EITHSGSTNYNPSLKS

SEQ ID NO: 4 - protein, 14 aa, synthetic construct
  REGION 1..14: note = Synthetic: HC CDR3 antibody F19
EIAVAGTGYYGMDV

SEQ ID NO: 5 - protein, 11 aa, synthetic construct
  REGION 1..11: note = Synthetic: LC CDR1 antibody F19
RASQGINSAFA

SEQ ID NO: 6 - protein, 7 aa, synthetic construct
  REGION 1..7: note = Synthetic: LC CDR2 antibody F19
DASSLES

SEQ ID NO: 7 - protein, 9 aa, synthetic construct
  REGION 1..9: note = Synthetic: LC CDR3 antibody F19
QQFNSYPLT

SEQ ID NO: 8 - protein, 448 aa, synthetic construct
  REGION 1..448: note = Synthetic: Heavy chain variable region antibody F19
QVQLQQWGAGLLKPSETLSLTCAVYGGSFSGYNWHWIRQPPGKGLEWIGEITHSGSTNYNPSLKSRVTISVDTSKNQFSLKLSSVTAADTAVYYCVREIAVAGTGYYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 9 - protein, 214 aa, synthetic construct
  REGION 1..214: note = Synthetic: Light chain variable region antibody F19
AIQLTQSPSSLSASVGDRVTITCRASQGINSAFAWYQQKPGKAPKLLIYDASSLESGVPSRFSGSGSGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 11 aa, synthetic construct
  REGION 1..11: note = Synthetic: Alternative LC CDR1 antibody F19
RASRGINSAFA

SEQ ID NO: 11 - protein, 7 aa, synthetic construct
  REGION 1..7: note = Synthetic: Alternative LC CDR2 antibody F19
DASSLES

SEQ ID NO: 12 - protein, 9 aa, synthetic construct
  REGION 1..9: note = Synthetic: Alternative LC CDR3 antibody F19
QQFNSYPLT

SEQ ID NO: 13 - protein, 11 aa, synthetic construct
  REGION 1..11: note = Synthetic: Alternative LC CDR1 antibody F19
RVSQGISSYLN

SEQ ID NO: 14 - protein, 7 aa, synthetic construct
  REGION 1..7: note = Synthetic: Alternative LC CDR2 antibody F19
SASNLQS

SEQ ID NO: 15 - protein, 8 aa, synthetic construct
  REGION 1..8: note = Synthetic: Alternative LC CDR3 antibody F19
ARTNAPPT

SEQ ID NO: 16 - protein, 11 aa, synthetic construct
  REGION 1..11: note = Synthetic: Alternative LC CDR1 antibody F19
RMSQGISSYLA

SEQ ID NO: 17 - protein, 7 aa, synthetic construct
  REGION 1..7: note = Synthetic: Alternative LC CDR2 antibody F19
AASTLQS

SEQ ID NO: 18 - protein, 9 aa, synthetic construct
  REGION 1..9: note = Synthetic: Alternative LC CDR3 antibody F19
QQYYSFPYT

SEQ ID NO: 19 - protein, 11 aa, synthetic construct
  REGION 1..11: note = Synthetic: Alternative LC CDR1 antibody F19
RASQGVSSYLA

SEQ ID NO: 20 - protein, 7 aa, synthetic construct
  REGION 1..7: note = Synthetic: Alternative LC CDR2 antibody F19
DASNRAT

SEQ ID NO: 21 - protein, 8 aa, synthetic construct
  REGION 1..8: note = Synthetic: Alternative LC CDR3 antibody F19
QQRSNWHP

SEQ ID NO: 22 - protein, 5 aa, synthetic construct
  REGION 1..5: note = Synthetic: HC CDR1 antibody E1
RFNMN

SEQ ID NO: 23 - protein, 17 aa, synthetic construct
  REGION 1..17: note = Synthetic: HC CDR2 antibody E1
YISSSSYTIYYADSVKG

SEQ ID NO: 24 - protein, 7 aa, synthetic construct
  REGION 1..7: note = Synthetic: HC CDR3 antibody E1
SIAAFDY

SEQ ID NO: 25 - protein, 11 aa, synthetic construct
  REGION 1..11: note = Synthetic: LC CDR1 antibody E1
RASQGISSALA

SEQ ID NO: 26 - protein, 7 aa, synthetic construct
  REGION 1..7: note = Synthetic: LC CDR2 antibody E1
DASSLES

SEQ ID NO: 27 - protein, 8 aa, synthetic construct
  REGION 1..8: note = Synthetic: LC CDR3 antibody E1
QQFNSYRT

SEQ ID NO: 28 - protein, 12 aa, synthetic construct
  REGION 1..12: note = Synthetic: Alternative LC CDR1 antibody E1
RASQSVSSSYLT

SEQ ID NO: 29 - protein, 7 aa, synthetic construct
  REGION 1..7: note = Synthetic: Alternative LC CDR2 antibody E1
GASSRAT

SEQ ID NO: 30 - protein, 9 aa, synthetic construct
  REGION 1..9: note = Synthetic: Alternative LC CDR3 antibody E1
QQYGSSMYT

SEQ ID NO: 31 - protein, 12 aa, synthetic construct
  REGION 1..12: note = Synthetic: Alternative LC CDR1 antibody E1
RASQSVSSSYLA

SEQ ID NO: 32 - protein, 7 aa, synthetic construct
  REGION 1..7: note = Synthetic: Alternative LC CDR2 antibody E1
GASNRAT

SEQ ID NO: 33 - protein, 9 aa, synthetic construct
  REGION 1..9: note = Synthetic: Alternative LC CDR3 antibody E1
QQYGSSPWT

SEQ ID NO: 34 - protein, 5 aa, synthetic construct
  REGION 1..5: note = Synthetic: HC CDR1 antibody E13
NAWMS

SEQ ID NO: 35 - protein, 19 aa, synthetic construct
  REGION 1..19: note = Synthetic: HC CDR2 antibody E13
RIKSKIDGGTTDYAAPVKG

SEQ ID NO: 36 - protein, 8 aa, synthetic construct
  REGION 1..8: note = Synthetic: HC CDR3 antibody E13
AMAGAFGF

SEQ ID NO: 37 - protein, 12 aa, synthetic construct
  REGION 1..12: note = Synthetic: LC CDR1 antibody E13
RASQSVSSSYLA

SEQ ID NO: 38 - protein, 7 aa, synthetic construct
  REGION 1..7: note = Synthetic: LC CDR2 antibody E13
GASSRAT

SEQ ID NO: 39 - protein, 10 aa, synthetic construct
  REGION 1..10: note = Synthetic: LC CDR3 antibody E13
QQYGSSPMYT

SEQ ID NO: 40 - protein, 7 aa, synthetic construct
  REGION 1..7: note = Synthetic: HC CDR1 antibody E63
SGGYYWS

SEQ ID NO: 41 - protein, 16 aa, synthetic construct
  REGION 1..16: note = Synthetic: HC CDR2 antibody E63
YIYYSGSTNYNPSLKS

SEQ ID NO: 42 - protein, 12 aa, synthetic construct
  REGION 1..12: note = Synthetic: HC CDR3 antibody E63
WITMFRGVGFDP

SEQ ID NO: 43 - protein, 11 aa, synthetic construct
  REGION 1..11: note = Synthetic: LC CDR1 antibody E63
RASQSIGSSLH

SEQ ID NO: 44 - protein, 7 aa, synthetic construct
  REGION 1..7: note = Synthetic: LC CDR2 antibody E63
YASQSFS

SEQ ID NO: 45 - protein, 9 aa, synthetic construct
  REGION 1..9: note = Synthetic: LC CDR3 antibody E63
RQSSSLPLT

SEQ ID NO: 46 - protein, 5 aa, synthetic construct
  REGION 1..5: note = Synthetic: HC CDR1 antibody F23
GYYWN

SEQ ID NO: 47 - protein, 11 aa, synthetic construct
  REGION 1..11: note = Synthetic: HC CDR2 antibody F23
EINQYNPSLKS

SEQ ID NO: 48 - protein, 14 aa, synthetic construct
  REGION 1..14: note = Synthetic: HC CDR3 antibody F23
EIAIADKGYYGLDV

SEQ ID NO: 49 - protein, 11 aa, synthetic construct
  REGION 1..11: note = Synthetic: LC CDR1 antibody F23
RASQGISSALA

SEQ ID NO: 50 - protein, 7 aa, synthetic construct
  REGION 1..7: note = Synthetic: LC CDR2 antibody F23
DASSLES

SEQ ID NO: 51 - protein, 9 aa, synthetic construct
  REGION 1..9: note = Synthetic: LC CDR3 antibody F23
QQFNSYPLT

SEQ ID NO: 52 - protein, 5 aa, synthetic construct
  REGION 1..5: note = Synthetic: HC CDR1
SYYIH

SEQ ID NO: 53 - protein, 14 aa, synthetic construct
  REGION 1..14: note = Synthetic: HC CDR2
PGSDITKYNEKFKG

SEQ ID NO: 54 - protein, 10 aa, synthetic construct
  REGION 1..10: note = Synthetic: HC CDR3
GISTYSAMDF

SEQ ID NO: 55 - protein, 11 aa, synthetic construct
  REGION 1..11: note = Synthetic: LC CDR1
KASQDVGTAVA

SEQ ID NO: 56 - protein, 7 aa, synthetic construct
  REGION 1..7: note = Synthetic: LC CDR2
WASTRHT

SEQ ID NO: 57 - protein, 9 aa, synthetic construct
  REGION 1..9: note = Synthetic: LC CDR3
QQYSSYPLT

SEQ ID NO: 58 - protein, 122 aa, synthetic construct
  REGION 1..122: note = Synthetic: HC variable region
QVQLVQSGAEVKKPGASVKVSCKASGYTFTSYYIHWVRQAPGQRLEWMGWIFPGSDITKYNEKFKGRVTITRDTSASTAYMELSSLRSEDTAVYYCAREDYGISTYSAMDFWGQGTLVTVSS

SEQ ID NO: 59 - protein, 108 aa, synthetic construct
  REGION 1..108: note = Synthetic: LC variable region
DIQLTQSPSFLSASVGDRVTITCKASQDVGTAVAWYQQKPGKAPKLLIYWASTRHTGVPSRFSGSGSGTEFTLTISSLQPEDFATYYCQQYSSYPLTFGQGTKVEIKR

SEQ ID NO: 60 - protein, 5 aa, synthetic construct
  REGION 1..5: note = Synthetic: HC CDR1 of 18E04
HFDIN

SEQ ID NO: 61 - protein, 17 aa, synthetic construct
  REGION 1..17: note = Synthetic: HC CDR2 of 18E04
WMNPDSDNTDYAQEFQG

SEQ ID NO: 62 - protein, 7 aa, synthetic construct
  REGION 1..7: note = Synthetic: HC CDR3 of 18E04
GGTTLDY

SEQ ID NO: 63 - protein, 11 aa, synthetic construct
  REGION 1..11: note = Synthetic: LC CDR1 of 18E04
SGDALPKKYAY

SEQ ID NO: 64 - protein, 7 aa, synthetic construct
  REGION 1..7: note = Synthetic: LC CDR2 of 18E04
EDSKRPS

SEQ ID NO: 65 - protein, 11 aa, synthetic construct
  REGION 1..11: note = Synthetic: LC CDR3 of 18E04
YSTDSSDNHVI

SEQ ID NO: 66 - protein, 5 aa, synthetic construct
  REGION 1..5: note = Synthetic: HC CDR1 of 98C07
DYYMS

SEQ ID NO: 67 - protein, 16 aa, synthetic construct
  REGION 1..16: note = Synthetic: HC CDR2 of 98C07
YISRSSFIYYSESVKG

SEQ ID NO: 68 - protein, 8 aa, synthetic construct
  REGION 1..8: note = Synthetic: HC CDR3 of 98C07
WELSPFDY

SEQ ID NO: 69 - protein, 11 aa, synthetic construct
  REGION 1..11: note = Synthetic: LC CDR1 of 98C07
RASQGISNYLA

SEQ ID NO: 70 - protein, 7 aa, synthetic construct
  REGION 1..7: note = Synthetic: LC CDR2 of 98C07
AASSLQS

SEQ ID NO: 71 - protein, 9 aa, synthetic construct
  REGION 1..9: note = Synthetic: LC CDR3 of 98C07
QQYNTYPFT

SEQ ID NO: 72 - protein, 5 aa, synthetic construct
  REGION 1..5: note = Synthetic: HC CDR1 of 1C02
YYGIS

SEQ ID NO: 73 - protein, 17 aa, synthetic construct
  REGION 1..17: note = Synthetic: HC CDR2 of 1C02
WISANSGNTNYAQKFQG

SEQ ID NO: 74 - protein, 8 aa, synthetic construct
  REGION 1..8: note = Synthetic: HC CDR3 of 1C02
GGVAVLEY

SEQ ID NO: 75 - protein, 11 aa, synthetic construct
  REGION 1..11: note = Synthetic: LC CDR1 of 1C02
WASQGISSYLA

SEQ ID NO: 76 - protein, 7 aa, synthetic construct
  REGION 1..7: note = Synthetic: LC CDR2 of 1C02
VASTLQS

SEQ ID NO: 77 - protein, 9 aa, synthetic construct
  REGION 1..9: note = Synthetic: LC CDR3 of 1C02
QQLKIYPLT

SEQ ID NO: 78 - protein, 5 aa, synthetic construct
  REGION 1..5: note = Synthetic: HC CDR1 of 1C06
DYYMN

SEQ ID NO: 79 - protein, 17 aa, synthetic construct
  REGION 1..17: note = Synthetic: HC CDR2 of 1C06
DISSRDNTIYYADSVKG

SEQ ID NO: 80 - protein, 14 aa, synthetic construct
  REGION 1..14: note = Synthetic: HC CDR3 of 1C06
ARERGFGDYFGMDV

SEQ ID NO: 81 - protein, 11 aa, synthetic construct
  REGION 1..11: note = Synthetic: LC CDR1 of 1C06
RASQDISSALA

SEQ ID NO: 82 - protein, 7 aa, synthetic construct
  REGION 1..7: note = Synthetic: LC CDR2 of 1C06
DASSLES

SEQ ID NO: 83 - protein, 9 aa, synthetic construct
  REGION 1..9: note = Synthetic: LC CDR3 of 1C06
QQFNTYPLT

SEQ ID NO: 84 - protein, 122 aa, synthetic construct
  REGION 1..122: note = Heavy chain variable region (VH) of antibody F19
QVQLQQWGAGLLKPSETLSLTCAVYGGSFSGYNWHWIRQPPGKGLEWIGEITHSGSTNYNPSLKSRVTISVDTSKNQFSLKLSSVTAADTAVYYCVREIAVAGTGYYGMDVWGQGTTVTVSS

SEQ ID NO: 85 - protein, 107 aa, synthetic construct
  REGION 1..107: note = Light chain variable region (VL) of antibody F19
AIQLTQSPSSLSASVGDRVTITCRASQGINSAFAWYQQKPGKAPKLLIYDASSLESGVPSRFSGSGSGTDFTLTISSLQPEDFATYYCQQFNSYPLTFGGGTKVEIK

(19) United States
(12) Patent Application Publication
(10) Pub. No.: US 2025/0002598 A1
(43) Pub. Date: Jan. 2, 2025

(54) Methods of Treating Asthma with Anti-LIGHT Antibodies

(71) Applicant: Avalo Therapeutics, Inc., Rockville, MD (US)

(72) Inventors: Garry A. Neil, Havertown, PA (US); H. Jeffrey Wilkins, Ambler, PA (US); Sheridan J. Carrington, Philadephia, PA (US)

(73) Assignee: Avalo Therapeutics, Inc., Rockville, MD (US)

(21) Appl. No.: 18/756,623

(22) Filed: Jun. 27, 2024

Related U.S. Application Data
(63) Continuation of application No. PCT/US2023/060064, filed on Jan. 4, 2023.
(60) Provisional application No. 63/396,308, filed on Aug. 9, 2022.
(60) Provisional application No. 63/296,786, filed on Jan. 5, 2022.

(51) Int. Cl. C07K 16/28

(52) U.S. Cl. CPC C07K 16/2875

(57) ABSTRACT

The present disclosure relates to methods of treating asthma, including non-eosinophilic asthma (NEA), with anti-LIGHT antibodies.

### CROSS-REFERENCE TO RELATED APPLICATIONS

This application is a continuation of International Patent Application No. PCT/US2023/060064, filed Jan. 4, 2023, which claims the benefit of priority to U.S. Provisional Application No. 63/296,786, filed Jan. 5, 2022, and U.S. Provisional Application No. 63/396,308, filed Aug. 9, 2022, the contents of each of which are incorporated herein by reference for all purposes.

### SEQUENCE LISTING

The instant application contains a Sequence Listing which has been submitted electronically in XML format and is hereby incorporated by reference in its entirety. Said XML copy, created on Jun. 20, 2024, is named “01118-0061-00US_Seq_List_ST26.xml” and is 109,208 bytes in size.

### FIELD OF THE INVENTION

The present disclosure relates to methods of treating asthma, including non-eosinophilic asthma (NEA), with anti-LIGHT antibodies.

### BACKGROUND OF THE INVENTION

Asthma is a chronic disease of the lungs characterized by airway inflammation causing swelling and excess mucous production, and as a result, patients experience difficulty breathing which can be life threatening in severe cases. (Boulet et al., Am J Respir Crit Care Med. 2000 October; 162(4 Pt 1): 1308-1313). The prevalence of asthma in the United States is estimated at 25 million. (Asthma and Allergy Foundation of America, Asthma facts and figures, https://www.aafa.org/asthma-facts/(accessed Jan. 3, 2022)). According to the CDC, more than 50% of patients with current asthma had uncontrolled asthma. (Centers for Disease Control and Prevention. AsthmaStats: Uncontrolled asthma among adults, 2016, https://www.cdc.gov/asthma/asthma_stats/uncontrolled-asthma-adults.htm (accessed Jan. 3, 2022)). Asthma is associated with environmental and/or host factors such as smoking cigarettes, pollution, infections, and obesity. Patients present with respiratory symptoms such as wheeze, shortness of breath, cough, and chest tightness.

There are classifications of asthma based on the inciting factors (allergic versus non-allergic) and the nature of the immune-inflammatory response (Th2 or low/no Th2). (Ray and Kolls, Trends Immunol. 2017 December; 38(12): 942-954; Wenzel et al., Am. J. Respir. Crit. Care Med. 1999; 160:1001-1008; Woodruff et al. Am. J. Respir. Crit. Care Med. 2009; 180(5): 388-95). An allergic or Th2 biased response is associated with eosinophilic classifications and related to type 2 cytokines IL-4, IL-5, and IL-13. (Lambrecht et al., Immunity. 2019; 50(4): 975-991). Non-allergic or low/absent Th2 response is associated with neutrophilic and pauci-granulocytic classifications, which as a group are called non-eosinophilic asthma and account for 40%-50% of all asthma patients. (Douwes et al., Thorax. 2002 July; 57(7): 643-8; Stokes and Casale, Ann. Allergy Asthma Immunol. 2016 August; 117(2): 121-5; McGrath et al, Am J Resp Crit Care Med. 2012; 185(6): 612-619; Jiang et al., Allergy Asthma Clin Immunol. 2021; 17(1): 45).

The long-term control of asthma is typically accomplished through the use of long-acting bronchodilators in combination with inhaled corticosteroids as needed. For patients specifically with severe allergic asthma, biologic agents have been approved for treatment which target IgE (omalizumab) or the Th2-related cytokine pathways of IL-4 and IL-5 (mepolizumab, resilizumab, benralizumab, and dupilumab). The cytokine pathways identified as contributing to NEA include IL-6, IL-8, IL-17, IFNγ, TNFα, and G-CSF. (Lambrecht et al., Immunity. 2019; 50 (4): 975-991). For NEA patients with severe disease, there are no approved agents related to these pathways and, as such, there remains a significant unmet medical need for these patients. Commonly used asthma drugs such as inhaled corticosteroids (ICS) may exacerbate NEA by causing increased neutrophil levels. Many NEA patients remain uncontrolled on existing medications. (Esteban-Gorgojo et al., J Asthma Allergy. 2018; 11:267-281).

LIGHT (acronym for “homologous to Lymphotoxin, exhibits Inducible expression and competes with HSV Glycoprotein D for binding to HVEM (herpesvirus entry mediator), a receptor expressed on T lymphocytes”), also known as TNFSF14 (tumor necrosis factor superfamily member 14) is an important regulatory cytokine.

LIGHT (TNFSF14) belongs to the tumor necrosis factor superfamily and is expressed by activated T cells, monocytes-macrophages and additional types of antigen presenting cells. LIGHT is considered one of the “Master Regulators” of the immune system and has a key role in the communication system which controls immune response. LIGHT has a dual mechanism of action; exerting its effects by activating both T cells and B cells as well as upregulating other inflammatory cytokines.

LIGHT activates two key receptors, herpesvirus entry mediator (HVEM) and lymphotoxin β receptor (LTBR), both expressed on lung epithelial cells. Early in infection LIGHT released from neutrophils and macrophages bind cellular receptors, which causes inflammatory cell infiltration, releasing high levels of TNF and additional pro-inflammatory cytokines. LIGHT also has a co-stimulatory role in T cell activation driving proinflammatory and tissue damaging effects. (Ware, Advances in experimental medicine and biology 647, 146-155 (2009); Ware, Immunological reviews 223, 186-201 (2008)). Therefore, LIGHT has roles in many immune-mediated pathologies such as Crohn's Disease, IBD, Rheumatoid arthritis, and fibrosis. An additional receptor for LIGHT is a decoy receptor (coined DcR3), which binds LIGHT and interferes with its activity by competing with receptor binding. (Steinberg et al., Seminars in immunopathology 31, 207-221 (2009); Wroblewski et al., Biochemical pharmacology 65, 657-667 (2003)). In hyper inflammation and cytokine storm conditions, DcR3 is likely to be overwhelmed, generating high DCR3-free (active) LIGHT.

Applicant has found that anti-LIGHT therapy may provide a therapeutic option for asthma patients, including poorly controlled NEA patients. For example, human bronchial epithelial (BEC) cells express receptors for LIGHT (LTBR) and upon LIGHT stimulation of BECs in vitro there is a resulting broad gene expression of proinflammatory mediators, which are resistant to corticosteroid treatment, consistent with the clinical presentation of NEA. (da Silva Antunes et al., J. Immunol, 2015; 195:2429-41). Soluble proinflammatory mediators such as IL-6, IL-8, OSM, and MCP-1 were also detected in this in vitro system. In the clinic, administration of an oral CXCR2 antagonist resulted in lowered sputum neutrophil counts and fewer mild exacerbations. (Nair et al., Clin Exp Allergy. 2012; 42(7): 1097-103). Elevated LIGHT levels were found to be negatively associated with lung function (forced expiratory volume in 1 second (FEV₁) and forced vital capacity (FVC)), in sputum of patients with asthma. (Romeo et al., J Allergy Clin Immunol. 2013; 131(2 Suppl): AB203). LIGHT was elevated overall in patients with high neutrophils. (Hastie et al., J Allergy Clin Immunol. 2010; 125(5): 1028-1036). Elevated LIGHT was associated with increased cellular infiltrate and levels of Th1 cytokines as well as reduced lung function in asthma. (Romeo et al., J Allergy Clin Immunol. 2013; 131(2 Suppl): AB203; Kowal et al., J Allergy Clin Immunol. 2019 February; 143 (2) supplement (Abstract); see also Doherty et al., Nat Med. 2011; 17(5): 596-603). Along with soluble LIGHT detected in sputum, others have observed elevated serum levels of DcR3 in asthma patients, which are even greater during asthma exacerbations suggesting a response to elevated LIGHT levels in relation to asthma and disease activity. (Kowal et al., J Allergy Clin Immunol. 2019 February; 143 (2) supplement (Abstract)).

### SUMMARY OF THE INVENTION

The present disclosure includes, for example, methods of treating subjects having asthma, including non-eosinophilic asthma (NEA), comprising administering an effective amount of an anti-LIGHT antibody to a subject in need thereof.

Embodiment 1. A method of treating asthma, including non-eosinophilic asthma (NEA), comprising administering an effective amount of an anti-LIGHT antibody to a subject in need thereof.

Embodiment 2. The method of embodiment 1, wherein the anti-LIGHT antibody comprises a heavy chain and a light chain that together comprise one of the following sets of CDR-H1, CDR-H2, CDR-H3, CDR-L1, CDR-L2, and CDR-L3 amino acid sequences:
(a) SEQ ID NOs: 2, 3, 4, 5, 6, and 7;
(b) SEQ ID NOs: 10, 11, 12, 13, 14, and 15;
(c) SEQ ID NOs: 16, 17, 18, 19, 20, and 21;
(d) SEQ ID NOs: 22, 23, 24, 25, 26, and 27;
(e) SEQ ID NOs: 28, 29, 30, 31, 32, and 33;
(f) SEQ ID NOs: 34, 35, 36, 37, 38, and 39;
(g) SEQ ID NOs: 40, 41, 42, 43, 44, and 45;
(h) SEQ ID NOs: 46, 47, 48, 49, 50, and 51; and
(i) SEQ ID NOs: 52, 53, 54, 55, 56, and 57.

Embodiment 3. The method of embodiment 1 or embodiment 2, wherein the anti-LIGHT antibody comprises a heavy chain and a light chain that together comprise the following set of CDR-H1, CDR-H2, CDR-H3, CDR-L1, CDR-L2, and CDR-L3 amino acid sequences: SEQ ID NOs: 2, 3, 4, 5, 6, and 7.

Embodiment 4. The method of any one of embodiments 1-3, wherein the subject has poorly controlled asthma as determined by an Asthma Control Questionnaire (ACQ) score ≥1.5.

Embodiment 5. The method of any one of embodiments 1-3, wherein the subject has poorly controlled asthma on a long-acting beta-agonist (LABA) and an inhaled corticosteroid (ICS).

Embodiment 6. The method of embodiment 5, wherein the LABA is salmeterol.

Embodiment 7. The method of embodiment 5 or embodiment 6, wherein the ICS is fluticasone.

Embodiment 8. The method of any one of embodiments 1-7, wherein the subject has experienced exacerbation of asthma within 25 months prior to administration of a first dose of the anti-LIGHT antibody.

Embodiment 9. The method of any one of embodiments 1-8, wherein the subject's blood eosinophil count <300 cells/u L.

Embodiment 10. The method of any one of embodiments 1-9, wherein the subject's blood eosinophil count <150 cells/μL.

Embodiment 11. The method of any one of embodiments 1-10, wherein the subject is on a LABA at the time of administration of a first dose of the anti-LIGHT antibody, and wherein the LABA is discontinued at about day 14 following administration of the first dose of the anti-LIGHT antibody.

Embodiment 12. The method of any one of embodiments 1-11, wherein the subject is on an ICS at the time of administration of a first dose of the anti-LIGHT antibody, and wherein the ICS is reduced by 50% at about day 28 following administration of the first dose of the anti-LIGHT antibody.

Embodiment 13. The method of embodiment 12, wherein the ICS is discontinued at about day 42 following administration of the first dose of the anti-LIGHT antibody.

Embodiment 14. The method of any one of embodiments 1-13, wherein the anti-LIGHT antibody is administered at a dose of 3-12 mg/kg, 3-11 mg/kg, 3-10 mg/kg, 3-9 mg/kg, 3-8 mg/kg, 3-7 mg/kg, 3-6 mg/kg, 3-5 mg/kg, or 3-4 mg/kg.

Embodiment 15. The method of any one of embodiments 1-13, wherein the anti-LIGHT antibody is administered at a dose of 6 mg/kg.

Embodiment 16. The method of any one of embodiments 1-13, wherein the anti-LIGHT antibody is administered at a dose of 8 mg/kg.

Embodiment 17. The method of any one of embodiments 1-13, wherein the anti-LIGHT antibody is administered at a dose of 100-1000 mg, 100-900 mg, 100-800 mg, 100-700 mg, 100-600 mg, 100-500 mg, 100-400 mg, 100-300 mg, or 100-200 mg.

Embodiment 18. The method of any one of embodiments 1-13, wherein the anti-LIGHT antibody is administered at a dose of about 600 mg.

Embodiment 19. The method of any one of embodiments 1-18, wherein the anti-LIGHT antibody is administered about every 14 days, about every 21 days, about every 28 days, about every 35 days, about every 42 days, about every 49 days, about every 56 days, or monthly.

Embodiment 20. The method of any one of embodiments 1-18, wherein the anti-LIGHT antibody is administered about every 28 days.

Embodiment 21. The method of any one of embodiments 1-18, wherein the anti-LIGHT antibody is administered monthly.

Embodiment 22. The method of any one of embodiments 1-13, wherein the anti-LIGHT antibody is administered at a dose of 600 mg every 28 days.

Embodiment 23. The method of any one of embodiments 1-13, wherein the anti-LIGHT antibody is administered at a dose of 600 mg monthly.

Embodiment 24. The method of any one of embodiments 1-23, wherein at least three doses of the anti-LIGHT antibody are administered.

Embodiment 25. The method of any one of embodiments 1-24, wherein the anti-LIGHT antibody is administered subcutaneously.

Embodiment 26. The method of any one of embodiments 1-24, wherein the anti-LIGHT antibody is administered intravenously.

Embodiment 27. The method of any one of embodiments 1-26, wherein the subject is human.

Embodiment 28. The method of any one of embodiments 1-26, wherein the subject is an adult.

Embodiment 29. The method of any one of embodiments 1-27, wherein the subject is a pediatric subject.

Embodiment 30. The method of any one of embodiments 1-29, wherein the anti-LIGHT antibody comprises a variable heavy chain (VH) comprising an amino acid sequence of SEQ ID NO: 84.

Embodiment 31. The method of any one of embodiments 1-30, wherein the anti-LIGHT antibody comprises a variable light chain (VL) comprising an amino acid sequence of SEQ ID NO: 85.

Embodiment 32. The method of any one of embodiments 1-31, wherein the anti-LIGHT antibody comprises a heavy chain comprising an amino acid sequence of SEQ ID NO: 8.

Embodiment 33. The method of any one of embodiments 1-32, wherein the anti-LIGHT antibody comprises a light chain comprising an amino acid sequence of SEQ ID NO: 9.

Embodiment 34. The method of any one of embodiments 1-33, wherein administration of the anti-LIGHT antibody increases the subject's time to an asthma related event.

Embodiment 35. The method of any one of embodiments 1-34, wherein administration of the anti-LIGHT antibody decreases the proportion of subjects in a population of subjects with an asthma related event.

Embodiment 36. The method of embodiment 34 or embodiment 35, wherein the asthma related event is ≥6 additional reliever puffs of short-acting beta agonist (SABA) compared to baseline in a 24-hour period on 2 consecutive days, wherein baseline SABA use is determined by the average use in the 7 days preceding administration of a first dose of the anti-LIGHT antibody.

Embodiment 37. The method of embodiment 34 or embodiment 35, wherein the asthma related event is increase in ICS dose ≥4 times than the dose at baseline, wherein baseline ICS dose is defined as the dosage the subject received during the 30 days leading up to administration of a first dose of the anti-LIGHT antibody.

Embodiment 38. The method of embodiment 34 or embodiment 35, wherein the asthma related event is a decrease in peak flow of 30% or more (compared to baseline) on 2 consecutive days of treatment, wherein baseline peak flow is determined by the average of measurements in the 7 days preceding administration of a first dose of the anti-LIGHT antibody.

Embodiment 39. The method of embodiment 34 or embodiment 35, wherein the asthma related event is an asthma exacerbation requiring the use of systemic corticosteroids for at least 3 days.

Embodiment 40. The method of embodiment 34 or embodiment 35, wherein the asthma related event is a hospitalization or emergency room visit because of an asthma exacerbation.

Embodiment 41. The method of any one of embodiments 1-40, wherein administration of the anti-LIGHT antibody increases the subject's forced expiratory volume in 1 second (FEV₁).

Embodiment 42. The method of any one of embodiments 1-41, wherein administration of the anti-LIGHT antibody decreases the subject's fractional exhaled nitric oxide (FeNO).

Embodiment 43. The method of any one of embodiments 1-42, wherein administration of the anti-LIGHT antibody decreases the subject's asthma control questionnaire (ACQ) score.

Embodiment 44. The method of any one of embodiments 1-43, wherein administration of the anti-LIGHT antibody increases the subject's Standardized Asthma Quality of Life Questionnaire score for 12 years and older (AQLQ(S)+12) score.

Embodiment 45. The method of any one of embodiments 1-44, wherein administration of the anti-LIGHT antibody decreases the subject's Asthma Symptom Diary score.

Embodiment 46. The method of any one of embodiments 1-45, wherein administration of the anti-LIGHT antibody improves the subject's European Quality of Life-5 Dimension 5 level Questionnaire score.

Embodiment 47. The method of any one of embodiments 1-46, wherein administration of the anti-LIGHT antibody improves the subject's Patient Global Impression of Change/Severity score.

Embodiment 48. The method of any one of embodiments 1-47, wherein administration of the anti-LIGHT antibody improves the subject's Clinician Global Impression of Improvement/Severity score.

Embodiment 49. The method of any one of embodiments 1-48, wherein administration of the anti-LIGHT antibody reduces the subject's incidence of SABA use.

Embodiment 50. The method of any one of embodiments 1-49, wherein the method further comprises assaying free LIGHT prior to, during, or after administration of the anti-LIGHT antibody.

Embodiment 51. The method of any one of embodiments 1-50, wherein the method further comprises assaying total LIGHT prior to, during, or after administration of the anti-LIGHT antibody.

Embodiment 52. The method of any one of embodiments 1-51, wherein the method further comprises assaying DcR3 prior to, during, or after administration of the anti-LIGHT antibody.

Embodiment 53. The method of any one of embodiments 1-52, wherein the subject has elevated free LIGHT.

Embodiment 54. The method of any one of embodiments 1-53, wherein administration of the anti-LIGHT antibody reduces serum free LIGHT in the subject.

Embodiment 55. Use of an anti-LIGHT antibody in the manufacture of a medicament for treating asthma, including non-eosinophilic asthma (NEA).

Embodiment 56. The use of embodiment 55, wherein the anti-LIGHT antibody comprises a heavy chain and a light chain that together comprise one of the following sets of CDR-H1, CDR-H2, CDR-H3, CDR-L1, CDR-L2, and CDR-L3 amino acid sequences:
(a) SEQ ID NOs: 2, 3, 4, 5, 6, and 7;
(b) SEQ ID NOs: 10, 11, 12, 13, 14, and 15;
(c) SEQ ID NOs: 16, 17, 18, 19, 20, and 21;
(d) SEQ ID NOs: 22, 23, 24, 25, 26, and 27;
(e) SEQ ID NOs: 28, 29, 30, 31, 32, and 33;
(f) SEQ ID NOs: 34, 35, 36, 37, 38, and 39;
(g) SEQ ID NOs: 40, 41, 42, 43, 44, and 45;
(h) SEQ ID NOs: 46, 47, 48, 49, 50, and 51; and
(i) SEQ ID NOs: 52, 53, 54, 55, 56, and 57.

Embodiment 57. The use of embodiment 55 or embodiment 56, wherein the anti-LIGHT antibody comprises a heavy chain and a light chain that together comprise the following set of CDR-H1, CDR-H2, CDR-H3, CDR-L1, CDR-L2, and CDR-L3 amino acid sequences: SEQ ID NOs: 2, 3, 4, 5, 6, and 7.

Embodiment 58. The use of any one of embodiments 55-57, wherein the subject has poorly controlled asthma as determined by an Asthma Control Questionnaire (ACQ) score ≥1.5.

Embodiment 59. The use of any one of embodiments 55-57, wherein the subject has poorly controlled asthma on a long-acting beta-agonist (LABA) and an inhaled corticosteroid (ICS).

Embodiment 60. The use of embodiment 59, wherein the LABA is salmeterol.

Embodiment 61. The use of embodiment 59 or embodiment 60, wherein the ICS is fluticasone.

Embodiment 62. The use of any one of embodiments 55-61, wherein the subject has experienced exacerbation of asthma within 25 months prior to administration of a first dose of the anti-LIGHT antibody.

Embodiment 63. The use of any one of embodiments 55-62, wherein the subject's blood eosinophil count <300 cells/u L.

Embodiment 64. The use of any one of embodiments 55-63, wherein the subject's blood eosinophil count <150 cells/μL.

Embodiment 65. The use of any one of embodiments 55-64, wherein the subject is on a LABA at the time of administration of a first dose of the anti-LIGHT antibody, and wherein the LABA is discontinued at about day 14 following administration of the first dose of the anti-LIGHT antibody.

Embodiment 66. The use of any one of embodiments 55-65, wherein the subject is on an ICS at the time of administration of a first dose of the anti-LIGHT antibody, and wherein the ICS is reduced by 50% at about day 28 following administration of the first dose of the anti-LIGHT antibody.

Embodiment 67. The use of embodiment 66, wherein the ICS is discontinued at about day 42 following administration of the first dose of the anti-LIGHT antibody.

Embodiment 68. The use of any one of embodiments 55-67, wherein the anti-LIGHT antibody is administered at a dose of 3-12 mg/kg, 3-11 mg/kg, 3-10 mg/kg, 3-9 mg/kg, 3-8 mg/kg, 3-7 mg/kg, 3-6 mg/kg, 3-5 mg/kg, or 3-4 mg/kg.

Embodiment 69. The use of any one of embodiments 55-67, wherein the anti-LIGHT antibody is administered at a dose of 6 mg/kg.

Embodiment 70. The use of any one of embodiments 55-67, wherein the anti-LIGHT antibody is administered at a dose of 8 mg/kg.

Embodiment 71. The use of any one of embodiments 55-67, wherein the anti-LIGHT antibody is administered at a dose of 100-1000 mg, 100-900 mg, 100-800 mg, 100-700 mg, 100-600 mg, 100-500 mg, 100-400 mg, 100-300 mg, or 100-200 mg.

Embodiment 72. The use of any one of embodiments 55-67, wherein the anti-LIGHT antibody is administered at a dose of about 600 mg.

Embodiment 73. The use of any one of embodiments 55-72, wherein the anti-LIGHT antibody is administered about every 14 days, about every 21 days, about every 28 days, about every 35 days, about every 42 days, about every 49 days, about every 56 days, or monthly.

Embodiment 74. The use of any one of embodiments 55-72, wherein the anti-LIGHT antibody is administered about every 28 days.

Embodiment 75. The use of any one of embodiments 55-72, wherein the anti-LIGHT antibody is administered monthly.

Embodiment 76. The use of any one of embodiments 55-67, wherein the anti-LIGHT antibody is administered at a dose of 600 mg every 28 days.

Embodiment 77. The use of any one of embodiments 55-67, wherein the anti-LIGHT antibody is administered at a dose of 600 mg monthly.

Embodiment 78. The use of any one of embodiments 55-77, wherein at least three doses of the anti-LIGHT antibody are administered.

Embodiment 79. The use of any one of embodiments 55-78, wherein the anti-LIGHT antibody is administered subcutaneously.

Embodiment 80. The use of any one of embodiments 55-78, wherein the anti-LIGHT antibody is administered intravenously.

Embodiment 81. The use of any one of embodiments 55-80, wherein the subject is human.

Embodiment 82. The use of any one of embodiments 55-81, wherein the subject is an adult.

Embodiment 83. The use of any one of embodiments 55-81, wherein the subject is a pediatric subject.

Embodiment 84. The use of any one of embodiments 55-83, wherein the anti-LIGHT antibody comprises a variable heavy chain (VH) comprising an amino acid sequence of SEQ ID NO: 84.

Embodiment 85. The use of any one of embodiments 55-84, wherein the anti-LIGHT antibody comprises a variable light chain (VL) comprising an amino acid sequence of SEQ ID NO: 85.

Embodiment 86. The use of any one of embodiments 55-85, wherein the anti-LIGHT antibody comprises a heavy chain comprising an amino acid sequence of SEQ ID NO: 8.

Embodiment 87. The use of any one of embodiments 55-86, wherein the anti-LIGHT antibody comprises a light chain comprising an amino acid sequence of SEQ ID NO: 9.

Embodiment 88. The use of any one of embodiments 55-87, wherein administration of the anti-LIGHT antibody increases the subject's time to an asthma related event.

Embodiment 89. The use of any one of embodiments 55-88, wherein administration of the anti-LIGHT antibody decreases the proportion of subjects in a population of subjects with an asthma related event.

Embodiment 90. The use of embodiment 88 or embodiment 89, wherein the asthma related event is ≥6 additional reliever puffs of short-acting beta agonist (SABA) compared to baseline in a 24-hour period on 2 consecutive days, wherein baseline SABA use is determined by the average use in the 7 days preceding administration of a first dose of the anti-LIGHT antibody.

Embodiment 91. The use of embodiment 88 or embodiment 89, wherein the asthma related event is increase in ICS dose ≥4 times than the dose at baseline, wherein baseline ICS dose is defined as the dosage the subject received during the 30 days leading up to administration of a first dose of the anti-LIGHT antibody.

Embodiment 92. The use of embodiment 88 or embodiment 89, wherein the asthma related event is a decrease in peak flow of 30% or more (compared to baseline) on 2 consecutive days of treatment, wherein baseline peak flow is determined by the average of measurements in the 7 days preceding administration of a first dose of the anti-LIGHT antibody.

Embodiment 93. The use of embodiment 88 or embodiment 89, wherein the asthma related event is an asthma exacerbation requiring the use of systemic corticosteroids for at least 3 days.

Embodiment 94. The use of embodiment 88 or embodiment 89, wherein the asthma related event is a hospitalization or emergency room visit because of an asthma exacerbation.

Embodiment 95. The use of any one of embodiments 55-94, wherein administration of the anti-LIGHT antibody increases the subject's forced expiratory volume in 1 second (FEV₁).

Embodiment 96. The use of any one of embodiments 55-95, wherein administration of the anti-LIGHT antibody decreases the subject's fractional exhaled nitric oxide (FeNO).

Embodiment 97. The use of any one of embodiments 55-96, wherein administration of the anti-LIGHT antibody decreases the subject's asthma control questionnaire (ACQ) score.

Embodiment 98. The use of any one of embodiments 55-97, wherein administration of the anti-LIGHT antibody increases the subject's Standardized Asthma Quality of Life Questionnaire score for 12 years and older (AQLQ(S)+12) score.

Embodiment 99. The use of any one of embodiments 55-98, wherein administration of the anti-LIGHT antibody decreases the subject's Asthma Symptom Diary score.

Embodiment 100. The use of any one of embodiments 55-99, wherein administration of the anti-LIGHT antibody improves the subject's European Quality of Life-5 Dimension 5 level Questionnaire score.

Embodiment 101. The use of any one of embodiments 55-100, wherein administration of the anti-LIGHT antibody improves the subject's Patient Global Impression of Change/Severity score.

Embodiment 102. The use of any one of embodiments 55-101, wherein administration of the anti-LIGHT antibody improves the subject's Clinician Global Impression of Improvement/Severity score.

Embodiment 103. The use of any one of embodiments 55-102, wherein administration of the anti-LIGHT antibody reduces the subject's incidence of SABA use.

Embodiment 104. The use of any one of embodiments 55-103, wherein the method further comprises assaying free LIGHT prior to, during, or after administration of the anti-LIGHT antibody.

Embodiment 105. The use of any one of embodiments 55-104, wherein the method further comprises assaying total LIGHT prior to, during, or after administration of the anti-LIGHT antibody.

Embodiment 106. The use of any one of embodiments 55-105, wherein the method further comprises assaying DcR3 prior to, during, or after administration of the anti-LIGHT antibody.

Embodiment 107. The use of any one of embodiments 55-106, wherein the subject has elevated free LIGHT.

Embodiment 108. The use of any one of embodiments 55-107, wherein administration of the anti-LIGHT antibody reduces serum free LIGHT in the subject.

Embodiment 109. An anti-LIGHT antibody for use in the treatment of asthma, including non-eosinophilic asthma (NEA).

Embodiment 110. The antibody for use of embodiment 109, wherein the anti-LIGHT antibody comprises a heavy chain and a light chain that together comprise one of the following sets of CDR-H1, CDR-H2, CDR-H3, CDR-L1, CDR-L2, and CDR-L3 amino acid sequences:
(a) SEQ ID NOs: 2, 3, 4, 5, 6, and 7;
(b) SEQ ID NOs: 10, 11, 12, 13, 14, and 15;
(c) SEQ ID NOs: 16, 17, 18, 19, 20, and 21;
(d) SEQ ID NOs: 22, 23, 24, 25, 26, and 27;
(e) SEQ ID NOs: 28, 29, 30, 31, 32, and 33;
(f) SEQ ID NOs: 34, 35, 36, 37, 38, and 39;
(g) SEQ ID NOs: 40, 41, 42, 43, 44, and 45;
(h) SEQ ID NOs: 46, 47, 48, 49, 50, and 51; and
(i) SEQ ID NOs: 52, 53, 54, 55, 56, and 57.

Embodiment 111. The antibody for use of embodiment 109 or embodiment 110, wherein the anti-LIGHT antibody comprises a heavy chain and a light chain that together comprise the following set of CDR-H1, CDR-H2, CDR-H3, CDR-L1, CDR-L2, and CDR-L3 amino acid sequences: SEQ ID NOs: 2, 3, 4, 5, 6, and 7.

Embodiment 112. The antibody for use of any one of embodiments 109-111, wherein the subject has poorly controlled asthma as determined by an Asthma Control Questionnaire (ACQ) score ≥1.5.

Embodiment 113. The antibody for use of any one of embodiments 109-111, wherein the subject has poorly controlled asthma on a long-acting beta-agonist (LABA) and an inhaled corticosteroid (ICS).

Embodiment 114. The antibody for use of embodiment 113, wherein the LABA is salmeterol.

Embodiment 115. The antibody for use of embodiment 113 or embodiment 114, wherein the ICS is fluticasone.

Embodiment 116. The antibody for use of any one of embodiments 109-115, wherein the subject has experienced exacerbation of asthma within 25 months prior to administration of a first dose of the anti-LIGHT antibody.

Embodiment 117. The antibody for use of any one of embodiments 109-116, wherein the subject's blood eosinophil count <300 cells/μL.

Embodiment 118. The antibody for use of any one of embodiments 109-117, wherein the subject's blood eosinophil count <150 cells/μL.

Embodiment 119. The antibody for use of any one of embodiments 109-118, wherein the subject is on a LABA at the time of administration of a first dose of the anti-LIGHT antibody, and wherein the LABA is discontinued at about day 14 following administration of the first dose of the anti-LIGHT antibody.

Embodiment 120. The antibody for use of any one of embodiments 109-119, wherein the subject is on an ICS at the time of administration of a first dose of the anti-LIGHT antibody, and wherein the ICS is reduced by 50% at about day 28 following administration of the first dose of the anti-LIGHT antibody.

Embodiment 121. The antibody for use of embodiment 120, wherein the ICS is discontinued at about day 42 following administration of the first dose of the anti-LIGHT antibody.

Embodiment 122. The antibody for use of any one of embodiments 109-121, wherein the anti-LIGHT antibody is administered at a dose of 3-12 mg/kg, 3-11 mg/kg, 3-10 mg/kg, 3-9 mg/kg, 3-8 mg/kg, 3-7 mg/kg, 3-6 mg/kg, 3-5 mg/kg, or 3-4 mg/kg.

Embodiment 123. The antibody for use of any one of embodiments 109-121, wherein the anti-LIGHT antibody is administered at a dose of 6 mg/kg.

Embodiment 124. The antibody for use of any one of embodiments 109-121, wherein the anti-LIGHT antibody is administered at a dose of 8 mg/kg.

Embodiment 125. The antibody for use of any one of embodiments 109-121, wherein the anti-LIGHT antibody is administered at a dose of 100-1000 mg, 100-900 mg, 100-800 mg, 100-700 mg, 100-600 mg, 100-500 mg, 100-400 mg, 100-300 mg, or 100-200 mg.

Embodiment 126. The antibody for use of any one of embodiments 109-121, wherein the anti-LIGHT antibody is administered at a dose of about 600 mg.

Embodiment 127. The antibody for use of any one of embodiments 109-126, wherein the anti-LIGHT antibody is administered about every 14 days, about every 21 days, about every 28 days, about every 35 days, about every 42 days, about every 49 days, about every 56 days, or monthly.

Embodiment 128. The antibody for use of any one of embodiments 109-126, wherein the anti-LIGHT antibody is administered about every 28 days.

Embodiment 129. The antibody for use of any one of embodiments 109-126, wherein the anti-LIGHT antibody is administered monthly.

Embodiment 130. The antibody for use of any one of embodiments 109-121, wherein the anti-LIGHT antibody is administered at a dose of 600 mg every 28 days.

Embodiment 131. The antibody for use of any one of embodiments 109-121, wherein the anti-LIGHT antibody is administered at a dose of 600 mg monthly.

Embodiment 132. The antibody for use of any one of embodiments 109-131, wherein at least three doses of the anti-LIGHT antibody are administered.

Embodiment 133. The antibody for use of any one of embodiments 109-132, wherein the anti-LIGHT antibody is administered subcutaneously.

Embodiment 134. The antibody for use of any one of embodiments 109-132, wherein the anti-LIGHT antibody is administered intravenously.

Embodiment 135. The antibody for use of any one of embodiments 109-134, wherein the subject is human.

Embodiment 136. The antibody for use of any one of embodiments 109-135, wherein the subject is an adult.

Embodiment 137. The antibody for use of any one of embodiments 109-135, wherein the subject is a pediatric subject.

Embodiment 138. The antibody for use of any one of embodiments 109-137, wherein the anti-LIGHT antibody comprises a variable heavy chain (VH) comprising an amino acid sequence of SEQ ID NO: 84.

Embodiment 139. The antibody for use of any one of embodiments 109-138, wherein the anti-LIGHT antibody comprises a variable light chain (VL) comprising an amino acid sequence of SEQ ID NO: 85.

Embodiment 140. The antibody for use of any one of embodiments 109-139, wherein the anti-LIGHT antibody comprises a heavy chain comprising an amino acid sequence of SEQ ID NO: 8.

Embodiment 141. The antibody for use of any one of embodiments 109-140, wherein the anti-LIGHT antibody comprises a light chain comprising an amino acid sequence of SEQ ID NO: 9.

Embodiment 142. The antibody for use of any one of embodiments 109-141, wherein administration of the anti-LIGHT antibody increases the subject's time to an asthma related event.

Embodiment 143. The antibody for use of any one of embodiments 109-142, wherein administration of the anti-LIGHT antibody decreases the proportion of subjects in a population of subjects with an asthma related event.

Embodiment 144. The antibody for use of embodiment 142 or embodiment 143, wherein the asthma related event is ≥6 additional reliever puffs of short-acting beta agonist (SABA) compared to baseline in a 24-hour period on 2 consecutive days, wherein baseline SABA use is determined by the average use in the 7 days preceding administration of a first dose of the anti-LIGHT antibody.

Embodiment 145. The antibody for use of embodiment 142 or embodiment 143, wherein the asthma related event is increase in ICS dose ≥4 times than the dose at baseline, wherein baseline ICS dose is defined as the dosage the subject received during the 30 days leading up to administration of a first dose of the anti-LIGHT antibody.

Embodiment 146. The antibody for use of embodiment 142 or embodiment 143, wherein the asthma related event is a decrease in peak flow of 30% or more (compared to baseline) on 2 consecutive days of treatment, wherein baseline peak flow is determined by the average of measurements in the 7 days preceding administration of a first dose of the anti-LIGHT antibody.

Embodiment 147. The antibody for use of embodiment 142 or embodiment 143, wherein the asthma related event is an asthma exacerbation requiring the use of systemic corticosteroids for at least 3 days.

Embodiment 148. The antibody for use of embodiment 142 or embodiment 143, wherein the asthma related event is a hospitalization or emergency room visit because of an asthma exacerbation.

Embodiment 149. The antibody for use of any one of embodiments 109-148, wherein administration of the anti-LIGHT antibody increases the subject's forced expiratory volume in 1 second (FEV₁).

Embodiment 150. The antibody for use of any one of embodiments 109-149, wherein administration of the anti-LIGHT antibody decreases the subject's fractional exhaled nitric oxide (FeNO).

Embodiment 151. The antibody for use of any one of embodiments 109-150, wherein administration of the anti-LIGHT antibody decreases the subject's asthma control questionnaire (ACQ) score.

Embodiment 152. The antibody for use of any one of embodiments 109-151, wherein administration of the anti-LIGHT antibody increases the subject's Standardized Asthma Quality of Life Questionnaire score for 12 years and older (AQLQ(S)+12) score.

Embodiment 153. The antibody for use of any one of embodiments 109-152, wherein administration of the anti-LIGHT antibody decreases the subject's Asthma Symptom Diary score.

Embodiment 154. The antibody for use of any one of embodiments 109-153, wherein administration of the anti-LIGHT antibody improves the subject's European Quality of Life-5 Dimension 5 level Questionnaire score.

Embodiment 155. The antibody for use of any one of embodiments 109-154, wherein administration of the anti-LIGHT antibody improves the subject's Patient Global Impression of Change/Severity score.

Embodiment 156. The antibody for use of any one of embodiments 109-155, wherein administration of the anti-LIGHT antibody improves the subject's Clinician Global Impression of Improvement/Severity score.

Embodiment 157. The antibody for use of any one of embodiments 109-156, wherein administration of the anti-LIGHT antibody reduces the subject's incidence of SABA use.

Embodiment 158. The antibody for use of any one of embodiments 109-157, wherein the method further comprises assaying free LIGHT prior to, during, or after administration of the anti-LIGHT antibody.

Embodiment 159. The antibody for use of any one of embodiments 109-158, wherein the method further comprises assaying total LIGHT prior to, during, or after administration of the anti-LIGHT antibody.

Embodiment 160. The antibody for use of any one of embodiments 109-159, wherein the method further comprises assaying DcR3 prior to, during, or after administration of the anti-LIGHT antibody.

Embodiment 161. The antibody for use of any one of embodiments 109-160, wherein the subject has elevated free LIGHT.

Embodiment 162. The antibody for use of any one of embodiments 109-161, wherein administration of the anti-LIGHT antibody reduces serum free LIGHT in the subject.

### BRIEF DESCRIPTION OF THE DRAWINGS

FIG. 1 shows a clinical study design for treating patients with asthma, including NEA, with Antibody A. Antibody A refers to an anti-LIGHT antibody, wherein the anti-LIGHT antibody comprises the following six CDRs: a heavy chain CDR1 having an amino acid sequence of SEQ ID NO: 2; a heavy chain CDR2 having an amino acid sequence of SEQ ID NO: 3; a heavy chain CDR3 having an amino acid sequence of SEQ ID NO: 4; a light chain CDR1 having an amino acid sequence of SEQ ID NO: 5; a light chain CDR2 having an amino acid sequence of SEQ ID NO: 6; and a light chain CDR3 having an amino acid sequence of SEQ ID NO: 7.

### DETAILED DESCRIPTION OF THE INVENTION

The following definitions are provided to facilitate an understanding of the invention. They are not intended to limit the invention in any way.

### Definitions

For purposes of the present invention, “a” or “an” entity refers to one or more of that entity; for example, “a cDNA” refers to one or more cDNA or at least one cDNA. As such, the terms “a” or “an,” “one or more” and “at least one” can be used interchangeably herein. It is also noted that the terms “comprising,” “including,” and “having” can be used interchangeably. Furthermore, a compound “selected from the group consisting of” refers to one or more of the compounds in the list that follows, including mixtures (i.e. combinations) of two or more of the compounds. According to the present invention, an “isolated,” or “biologically pure” molecule is a compound that has been removed from its natural milieu. As such, the terms “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

“LIGHT” or “TNFSF14” herein refers to a specific member protein of the tumor necrosis factor superfamily that is expressed by activated T cells, monocytes-macrophages and additional types of antigen presenting cells. “LIGHT” is an acronym for “homologous to Lymphotoxin, exhibits Inducible expression and competes with HSV Glycoprotein D for binding to HVEM (herpesvirus entry mediator), a receptor expressed on T lymphocytes.”

“Free LIGHT” or “free (active) LIGHT” herein refers to non-bound form LIGHT (e.g., LIGHT bound to DcR3), which is the active form of LIGHT. In humans, free LIGHT is neutralized (inactivated) by DcR3, a unique soluble member of the TNFR superfamily, which binds LIGHT in high affinity and inhibits its interactions with two TNF receptors, HVEM and LTBR. “Bound LIGHT,” or the like, refers to LIGHT that is bound to a natural ligand, optionally wherein the natural ligand is HVEM, LTBR, or DcR3. “Total LIGHT,” or the like, refers to the total amount of free LIGHT and bound LIGHT.

“Elevated free LIGHT” as used herein refers to a level of free LIGHT detected in a subject that is higher than a normal control. The normal control can be determined by those of skill in the art as applicable to the particular situation. In some instances, the normal control is an industry standard agreed upon by those of skill as being a level or range of levels that is typical of an individual without a LIGHT-associated condition. In some instances, the normal control is a reference level of LIGHT from the same individual taken at a time point, and whether the subject has elevated LIGHT is determined based on a sample from that same individual taken at a different, typically later, time point.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. As used herein, the term refers to a molecule comprising at least complementarity-determining region (CDR) 1, CDR2, and CDR3 of a heavy chain and at least CDR1, CDR2, and CDR3 of a light chain, wherein the molecule is capable of binding to antigen. The term antibody includes, but is not limited to, fragments that are capable of binding antigen, such as Fv, single-chain Fv (scFv), Fab, Fab′, and (Fab′)₂. The term antibody also includes, but is not limited to, chimeric antibodies, humanized antibodies, human antibodies, and antibodies of various species such as mouse, cynomolgus monkey, etc.

The term “heavy chain” refers to a polypeptide comprising at least a heavy chain variable region, with or without a leader sequence. In some embodiments, a heavy chain comprises at least a portion of a heavy chain constant region. The term “full-length heavy chain” refers to a polypeptide comprising a heavy chain variable region and a heavy chain constant region, with or without a leader sequence.

The term “heavy chain variable region” refers to a region comprising a heavy chain complementary determining region (CDR) 1, framework region (FR) 2, CDR2, FR3, and CDR3 of the heavy chain. In some embodiments, a heavy chain variable region also comprises at least a portion of an FR1 and/or at least a portion of an FR4. In some embodiments, a heavy chain CDR1 corresponds to Kabat residues 31 to 35; a heavy chain CDR2 corresponds to Kabat residues 50 to 65; and a heavy chain CDR3 corresponds to Kabat residues 95 to 102. See, e.g., Kabat Sequences of Proteins of Immunological Interest (1987 and 1991, NIH, Bethesda, Md.).

The term “light chain” refers to a polypeptide comprising at least a light chain variable region, with or without a leader sequence. In some embodiments, a light chain comprises at least a portion of a light chain constant region. The term “full-length light chain” refers to a polypeptide comprising a light chain variable region and a light chain constant region, with or without a leader sequence. The term “light chain variable region” refers to a region comprising a light chain CDR1, FR2, HVR2, FR3, and HVR3. In some embodiments, a light chain variable region also comprises an FR1 and/or an FR4. In some embodiments, a light chain CDR1 corresponds to Kabat residues 24 to 34; a light chain CDR2 corresponds to Kabat residues 50 to 56; and a light chain CDR3 corresponds to Kabat residues 89 to 97. See, e.g., Kabat Sequences of Proteins of Immunological Interest (1987 and 1991, NIH, Bethesda, Md.).

A “chimeric antibody” refers to an antibody in which a portion of the heavy and/or light chain is derived from a particular source or species, while the remainder of the heavy and/or light chain is derived from a different source or species. In some embodiments, a chimeric antibody refers to an antibody comprising at least one variable region from a first species (such as mouse, rat, cynomolgus monkey, etc.) and at least one constant region from a second species (such as human, cynomolgus monkey, etc.). In some embodiments, a chimeric antibody comprises at least one mouse variable region and at least one human constant region. In some embodiments, a chimeric antibody comprises at least one cynomolgus variable region and at least one human constant region. In some embodiments, all of the variable regions of a chimeric antibody are from a first species and all of the constant regions of the chimeric antibody are from a second species.

A “humanized antibody” refers to an antibody in which at least one amino acid in a framework region of a non-human variable region has been replaced with the corresponding amino acid from a human variable region. In some embodiments, a humanized antibody comprises at least one human constant region or fragment thereof. In some embodiments, a humanized antibody is an Fab, an scFv, a (Fab′) 2, etc.

A “human antibody” as used herein refers to antibodies produced in humans, antibodies produced in non-human animals that comprise human immunoglobulin genes, such as XenoMouse®, and antibodies selected using in vitro methods, such as phage display, wherein the antibody repertoire is based on a human immunoglobulin sequences.

The term “leader sequence” refers to a sequence of amino acid residues located at the N terminus of a polypeptide that facilitates secretion of a polypeptide from a mammalian cell. A leader sequence may be cleaved upon export of the polypeptide from the mammalian cell, forming a mature protein. Leader sequences may be natural or synthetic, and they may be heterologous or homologous to the protein to which they are attached.

“Percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

The terms “inhibition” or “inhibit” refer to a decrease or cessation of any event (such as protein ligand binding) or to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. It is not necessary that the inhibition or reduction be complete. For example, in certain embodiments, by “reduce” or “inhibit” is meant the ability to cause an overall decrease of 20% or greater. In another embodiment, by “reduce” or “inhibit” is meant the ability to cause an overall decrease of 50% or greater. In yet another embodiment, by “reduce” or “inhibit” is meant the ability to cause an overall decrease of 75%, 85%, 90%, 95%, or greater.

“Sample” or “subject sample” or “biological sample” generally refers to a sample which may be tested for a particular molecule. Samples may include but are not limited to cells, body fluids, including blood, serum, plasma, urine, saliva, stool, tears, pleural fluid and the like.

The terms “agent” and “test compound” are used interchangeably herein and denote a chemical compound, a mixture of chemical compounds, a biological macromolecule, or an extract made from biological materials such as bacteria, plants, fungi, or animal (particularly mammalian) cells or tissues. Biological macromolecules include siRNA, shRNA, antisense oligonucleotides, peptides, peptide/DNA complexes, and any nucleic acid based molecule which exhibits the capacity to modulate the activity of the SNP containing nucleic acids described herein or their encoded proteins. Agents are evaluated for potential biological activity by inclusion in screening assays described hereinbelow.

A “subject” can be mammalian. In any of the embodiments involving a subject, the subject can be human. In any of the embodiments involving a subject, the subject can be a cow, pig, monkey, sheep, dog, cat, fish, or poultry.

A “pediatric” subject herein is a human of less than 18 years of age, whereas an “adult” subject is 18 years or older.

“Treatment” or “treat” refers to both therapeutic treatment and prophylactic or preventative measures. Those in need of treatment include those already with the disorder as well as those prone to have the disorder or those in which the disorder is to be prevented. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The term “effective amount” or “therapeutically effective amount” refers to an amount of a drug effective for treatment of a disease or disorder in a subject, such as to partially or fully relieve one or more symptoms. In some embodiments, an effective amount refers to an amount effective, at dosages and for periods of time necessary, to achieve the desired therapeutic or prophylactic result.

The term “non-eosinophilic asthma” or “NEA” refers to NEA characterized by airway inflammation with the absence, or a low number of, eosinophils, subsequent to activation of non-predominant type 2 immunologic pathways. In other words, NEA typically does not have the features of T-helper cell type 2 asthma and generally is based on the presence of neutrophils in sputum or the absence (or normal levels) of eosinophils or other T2 markers in sputum, biopsy samples, or blood. It may be defined by an eosinophil count in the sputum, such as <2% or <3%. It may also be defined as a concentration of eosinophils in the blood below a certain threshold, such as <300 eosinophils/μL. It can also include sub phenotypes. NEA patients typically do not respond well to inhaled corticosteroids.

The term “FEV₁” refers to forced expiratory volume 1, a measure of how much air a person can exhale during a forced breath. FEV₁ may be measured during spirometry, using an instrument called a spirometer. FEV₁ may be used to describe the degree of airway obstruction caused by asthma. FEV₁ may be calculated by converting the spirometer reading to a percentage of what would be predicted as normal (i.e., compared to the standard or expected FEV₁ score based on a healthy person, taking into account gender, height, and race). A person who has asthma typically has a lower FEV₁ than a healthy person. Sometimes, FEV₁ is included as part of an FEV₁/FVC ratio. FVC refers to forced vital capacity, the full amount of air that can be exhaled with effort in a complete breath.

The term “peak flow” refers to a measure of how fast air comes out of the lungs when one exhales forcefully after inhaling fully. It is also called “peak expiratory flow” (“PEF”) or peak expiratory flow rate (“PEFR”). Peak flow may be measured with a device. A commonly used device to measure peak flow is a peak flow meter (“PFM”). A person's “normal” peak flow may be based on the person's age, height, sex, and race. Peak flow is commonly split into three zones-green, yellow, and red. The green zone is 80 to 100 percent of the person's usual or “normal” peak flow rate and signals all clear. A reading in this zone means that the person's asthma is in good control. The yellow zone is 50 to 80 percent of a person's usual or “normal” peak flow rate and signals caution. This zone indicates that the person's airways are narrowing, and that the person needs to take action. The red zone is less than 50 percent of the person's usual or “normal” peak flow rate and signals a medical alert. This zone indicates that there is severe airway narrowing.

The term “FeNO” refers to fraction of exhaled nitric oxide or fractional exhaled nitric oxide; in other words, level of nitric oxide during exhalation. FeNO is a biomarker of bronchial or airway inflammation. FeNO is produced by airway epithelial cells in response to inflammatory cytokines. FeNO levels in healthy adults range from 2 to 30 parts per billion (ppb). An exemplary assay for measuring FeNO is by using a NIOX® instrument by Circassia AB. The assessment may be conducted prior to spirometry and following a fast of at least an hour.

The term “LABA” refers to long-acting beta agonist. LABAs are bronchodilators. Examples of LABAs include, but are not limited to, salmeterol (e.g., Serevent™), formoterol (e.g., Foradil™), and the like. LABAs may be administered as part of a combination therapy with an inhaled corticosteroid (ICS). Such combination therapies include, for example: ADVAIR® (GlaxoSmithKline) (fluticasone+salmeterol), SYMBICORT® (AstraZeneca) (budesonide+formoterol), DULERA® (Organon) (mometasone+formoterol).

The term “ICS” refers to inhaled corticosteroids. Inhaled corticosteroids help control asthma symptoms. Examples of inhaled corticosteroids include, but are not limited to, fluticasone (e.g., fluticasone propionate, e.g., Flovent™), budesonide, mometasone (e.g., mometasone furoate, e.g., Asmanex™), flunisolide (e.g., Acrobid™), 5 dexamethasone acetate/phenobarbital/theophylline (e.g., Azmacort™), beclomethasone dipropionate HFA (Qvar™), and the like. Inhaled corticosteroids may be administered as part of a combination therapy with a long-acting beta agonist (“LABA”). Such combination therapies include, for example: ADVAIR® (GlaxoSmithKline) (fluticasone+salmeterol), SYMBICORT® (AstraZeneca) (budesonide+formoterol), DULERA® (Organon) (mometasone+formoterol).

The term “SABA” refers to short-acting beta agonist. SABAs are also known as “quick-acting beta2-adrenergic receptor agonists” or “reliever medications” or “rescue medications.” They are typically used to provide quick relief of asthma symptoms. Examples of SABAs include, but are not limited to, albuterol (i.e., salbutamol, e.g., Proventil™, Ventolin™, ProAir™ and the like), levalbuterol (e.g., Xopenex™), pirbuterol (e.g., Maxair™), metaproterenol (e.g., Alupent™) and the like.

The term “LTA” refers to leukotriene receptor antagonist (LTA). Examples of LTAs include, but are not limited to, montelukast (e.g., Singulaire™), zafirlukast (e.g., Accolate™), and the like.

The term “ACQ” refers to the asthma control questionnaire. The ACQ is a simple, validated questionnaire that measures the adequacy of asthma control and change in asthma control which occurs either spontaneously or as a result of treatment. It has 7 items, some of which are scaled on a 7-point scale (there are 7 categories for FEV₁) and some of which are scaled on a 6-point scale (0=no impairment, 6=maximum impairment for symptoms and rescue use). Scores range between 0 (totally controlled) and 6 (severely uncontrolled). The questions are equally weighted and the ACQ score is the mean of the 7 responses to the questions and therefore between 0 (totally controlled) and 6 (severely uncontrolled). A score of 1.5 or more may indicate that the subject has inadequate asthma control. A lower score generally indicates better asthma control. A change in score of 0.5 on the ACQ can be considered clinically important. Shortened versions of the original ACQ are available. (Juniper E F, O'Byrne P M, Guyatt G H, Ferric P J, King D R. Eur Respir J 1999; 14:902-907; Juniper E F, Bousquet J, Abetz L, Bateman E D. Respiratory Medicine 2006(100): 616-621; Juniper E F, Svensson K, Mork A C, Stahl E. Respiratory Medicine 2005(99): 553-558).

An “AQLQ(S)+12” refers to a subject's Standardized Asthma Quality of Life Questionnaire score for 12 years and older score, also called “AQLQ 12+.” The AQLQ12+ was designed to measure the functional impairments that are most troublesome to people aged 12 years and older as a result of their asthma. The instrument is comprised of 32 items, each rated on 7-point Likert scales from 1 to 7. The AQLQ12+ has 4 domains. The domains and the number of items in each domain are as follows: Symptoms (12 items), Activity limitation (11 items), Emotional function (5 items), and Environmental Stimuli (4 items). A global score is calculated ranging from 0 to 7, and a score by domain. Higher scores indicate better quality of life. A score of 7=no impairment and a score of 1=severe impairment. The overall score is calculated as the mean response to all questions. The four domain scores (symptoms, activity limitations, emotional function, and environmental stimuli) are the means of the responses to the questions in each of the domains.

The “Asthma Symptom Diary Score” refers to the score on a 6-item daily measure of asthma symptom severity that assesses three core categories of asthma symptoms: breathing symptoms (difficulty breathing; wheezing; shortness of breath), chest symptoms (chest tightness; chest pain), and cough. The Asthma Symptom Diary is intended for twice daily completion and comprises a morning diary (for completion upon waking and referring to asthma symptoms during the nighttime) and an evening diary (for completion before going to bed and referring to asthma symptoms during the day). Patients rate the 6 symptoms at their worst during the respective timeframes using an 11-point numeric rating scale ranging from 0 (“None”) to 10 (“As bad as you can imagine”). (United States Food and Drug Administration. Clinical Outcome Assessments (COA) Qualification Program DDT COA #000006: Asthma Daytime Symptom Diary (ADSD) and Asthma Nighttime Symptom Diary (ANSD), available at https://www.fda.gov/drugs/clinical-outcome-assessment-coa-qualification-program/ddt-coa-000006-asthma-daily-symptom-diary-adsd).

The “European Quality of Life-5 Dimension 5 level Questionnaire score” also referred to as the “EQ5D-5L” or the “EQ-5D-5L” refers to a score from a test that consists of the EQ-5D descriptive system and the EQ visual analogue scale (EQ VAS). The descriptive system comprises five dimensions: mobility, self-care, usual activities, pain/discomfort, and anxiety/depression. Each dimension has 5 levels: no problems, slight problems, moderate problems, severe problems, and extreme problems. The EQ VAS records the subject's self-rated health on a vertical VAS, where the endpoints are labelled ‘The best health you can imagine’ and ‘The worst health you can imagine’ (EuroQol, Group. EQ-5D-5L: About. 2017, available at https://euroqol.org/cq-5d-instruments/eq-5d-51-about/EuroQol, 2017).

The “Clinician Global Impression of Improvement/Severity score” or “Clinical Global Impressions Scale score” or “CGI-I, CGI-S” refers to a global index that can be used to rate the severity of a specific condition (a single-state scale) from the clinician's perspective. The CGI-I is a single question scale asking the clinician to rate the overall status of the patient's specific condition on a 7-point scale since the beginning of the research study. The CGI-S is a single question scale asking the clinician to rate the current state of the patient's specific condition on a 7-point scale. For example, a CGI-S score of 1 may mean normal, not at all ill, a score of 2 may mean borderline ill, a score of 3 may mean mildly ill, a score of 4 may mean moderately ill, a score of 5 may mean markedly ill, a score of 6 may mean severely ill, and a score of 7 may mean that the patient is among the most extremely ill patients. For example, a CGI-I score of 1 may mean very much improved, a score of 2 may mean much improved, a score of 3 may mean minimally improved, a score of 4 may mean no change, a score of 5 may mean minimally worse, a score of 6 may mean much worse, and a CGI-I score of 7 may mean very much worse. See Guy W (ed) (1976) ECDEU assessment manual for psychopharmacology. US Department of Health, Education, and Welfare, Rockville, MD.

The “Patient Global Impression of Change/Severity score” or “PGI-C, PGI-S” scale refers to a global index that can be used to rate the severity of a specific condition (a single-state scale) from the patient's perspective. The PGI-C is a single question scale asking the patient to rate the overall status of their specific condition on a 7-point scale. The PGI-S is a single question scale asking the patient to rate current state of their specific condition on a 7-point scale. For example, a PGI-S score of 1 may mean normal, not at all ill, a score of 2 may mean borderline ill, a score of 3 may mean mildly ill, a score of 4 may mean moderately ill, a score of 5 may mean markedly ill, a score of 6 may mean severely ill, and a score of 7 may mean that the patient is among the most extremely ill patients. For example, a PGI-I score of 1 may mean very much improved, a score of 2 may mean much improved, a score of 3 may mean minimally improved, a score of 4 may mean no change, a score of 5 may mean minimally worse, a score of 6 may mean much worse, and a score of 7 may mean very much worse. See Guy W (cd) (1976) ECDEU assessment manual for psychopharmacology. US Department of Health, Education, and Welfare, Rockville, MD.

“Exacerbation of asthma” or “asthma exacerbation” refers to an increase in the severity and/or frequency and/or duration of one or more symptoms or indicia of asthma. It also includes any deterioration in the respiratory health of a subject that requires and or is treatable by a therapeutic intervention for asthma (such as, e.g., steroid treatment, inhaled corticosteroid treatment, hospitalization, etc.).

“Improved” herein is used to indicate that an asthma-associated parameter is quantified at a baseline time point and at a time point after administration of the anti-LIGHT antibody. The difference between the value of the parameter at a particular time point following initiation of treatment and the value of the parameter at a baseline time point is used to establish whether there has been an “improvement” in the asthma associated parameter. This can be an increase or decrease, depending on the specific parameter measured.

Methods of Treating Asthma, Including NEA, with Anti-LIGHT Antibody

In some embodiments, a method of treating a subject having asthma is provided comprising administering an anti-LIGHT antibody to a subject in need thereof. In some embodiments, the subject has poorly controlled asthma as determined by an Asthma Control Questionnaire (ACQ) score ≥1.5. In some embodiments, the subject has poorly controlled asthma on a long-acting beta-agonist (LABA), optionally wherein the LABA is salmeterol, and on an inhaled corticosteroid (ICS), optionally wherein the ICS is fluticasone. In some embodiments, the subject has experienced exacerbation of asthma within 25 months prior to administration of a first dose of the anti-LIGHT antibody. In some embodiments, the subject has experienced exacerbation of asthma within 24 months prior to a screening visit. In some embodiments, the subject's blood eosinophil count <300 cells/μL. In some embodiments, the subject's blood eosinophil count <250 cells/μL. In some embodiments, the subject's blood eosinophil count <150 cells/μL. In some embodiments, the subject is on a LABA at the time of administration of a first dose of the anti-LIGHT antibody, and the LABA is discontinued at about day 14 following administration of the first dose of the anti-LIGHT antibody. In some embodiments, the subject is on an ICS at the time of administration of a first dose of the anti-LIGHT antibody, and the ICS is reduced by 50% at about day 28 following administration of the first dose of the anti-LIGHT antibody. In some embodiments, the ICS is discontinued at about day 42 following the first administration of the anti-LIGHT antibody. In some embodiments, the anti-LIGHT antibody is administered at a dose of 3-12 mg/kg. In some embodiments, the anti-LIGHT antibody is administered at a dose of 3-11 mg/kg. In some embodiments, the anti-LIGHT antibody is administered at a dose of 3-10 mg/kg. In some embodiments, the anti-LIGHT antibody is administered at a dose of 3-9 mg/kg. In some embodiments, the anti-LIGHT antibody is administered at a dose of 3-8 mg/kg. In some embodiments, the anti-LIGHT antibody is administered at a dose of 3-7 mg/kg. In some embodiments, the anti-LIGHT antibody is administered at a dose of 3-6 mg/kg. In some embodiments, the anti-LIGHT antibody is administered at a dose of 3-5 mg/kg. In some embodiments, the anti-LIGHT antibody is administered at a dose of 3-4 mg/kg. In some embodiments, the anti-LIGHT antibody is administered at a dose of 6 mg/kg. In some embodiments, the anti-LIGHT antibody is administered at a dose of 7 mg/kg. In some embodiments, the anti-LIGHT antibody is administered at a dose of 8 mg/kg. In some embodiments, the anti-LIGHT antibody is administered at a dose of 100-1000 mg. In some embodiments, the anti-LIGHT antibody is administered at a dose of 100-900 mg. In some embodiments, the anti-LIGHT antibody is administered at a dose of 100-800 mg. In some embodiments, the anti-LIGHT antibody is administered at a dose of 100-700 mg. In some embodiments, the anti-LIGHT antibody is administered at a dose of 100-600 mg. In some embodiments, the anti-LIGHT antibody is administered at a dose of 100-500 mg. In some embodiments, the anti-LIGHT antibody is administered at a dose of 100-400 mg. In some embodiments, the anti-LIGHT antibody is administered at a dose of 100-300 mg. In some embodiments, the anti-LIGHT antibody is administered at a dose of 100-200 mg. In some embodiments, the anti-LIGHT antibody is administered at a dose of about 600 mg. In some embodiments, the anti-LIGHT antibody is administered about every 14 days. In some embodiments, the anti-LIGHT antibody is administered about every 21 days. In some embodiments, the anti-LIGHT antibody is administered about every 28 days. In some embodiments, the anti-LIGHT antibody is administered about every 35 days. In some embodiments, the anti-LIGHT antibody is administered about every 42 days. In some embodiments, the anti-LIGHT antibody is administered about every 49 days. In some embodiments, the anti-LIGHT antibody is administered about every 56 days. In some embodiments, the anti-LIGHT antibody is administered every four weeks. In some embodiments, the anti-LIGHT antibody is administered monthly. In some embodiments, the anti-LIGHT antibody is administered at a dose of 8 mg/kg every four weeks. In some embodiments, the anti-LIGHT antibody is administered at a dose of 8 mg/kg monthly. In some embodiments, the anti-LIGHT antibody is administered at a dose of 6 mg/kg every four weeks. In some embodiments, the anti-LIGHT antibody is administered at a dose of 6 mg/kg monthly. In some embodiments, the anti-LIGHT antibody is administered at a dose of 600 mg every 28 days. In some embodiments, the anti-LIGHT antibody is administered at a dose of 600 mg monthly. In some embodiments, the anti-LIGHT antibody is administered for twelve weeks. In some embodiments, at least three doses of the anti-LIGHT antibody are administered. In some embodiments, the anti-LIGHT antibody is administered subcutaneously. In some embodiments, the anti-LIGHT antibody is administered intravenously. In some embodiments, the subject is human. In some embodiments, the subject is an adult. In some embodiments, the subject is a pediatric subject. In some embodiments, administration of the anti-LIGHT antibody increases the subject's time to exacerbation of asthma. In some embodiments, administration of the anti-LIGHT antibody increases the subject's time to an asthma related event. In some embodiments, administration of the anti-LIGHT antibody decreases the proportion of subjects in a population of subjects with exacerbation of asthma. In some embodiments, administration of the anti-LIGHT antibody decreases the proportion of subjects in a population of subjects with an asthma-related event. In some embodiments, the asthma related event is ≥6 additional reliever puffs of short-acting beta agonist (SABA) compared to baseline in a 24-hour period on 2 consecutive days, wherein baseline SABA use is determined by the average use in the 7 days preceding administration of a first dose of the anti-LIGHT antibody. In some embodiments, the asthma related event is increase in ICS dose ≥4 times than the dose at baseline, wherein baseline ICS dose is defined as the dosage the subject received during the 30 days leading up to administration of a first dose of the anti-LIGHT antibody. In some embodiments, the asthma related event is a decrease in peak flow of 30% or more (compared to baseline) on 2 consecutive days of treatment, wherein baseline peak flow is determined by the average of measurements in the 7 days preceding administration of a first dose of the anti-LIGHT antibody. In some embodiments, the asthma related event is an asthma exacerbation requiring the use of systemic corticosteroids (tablets, suspension, or injection) for at least 3 days. In some embodiments, the asthma related event is a hospitalization or emergency room visit because of an asthma exacerbation. In some embodiments, administration of the anti-LIGHT antibody increases the subject's forced expiratory volume in 1 second (FEV₁). In some embodiments, administration of the anti-LIGHT antibody decreases the subject's fractional exhaled nitric oxide (FeNO). In some embodiments, administration of the anti-LIGHT antibody decreases the subject's asthma control questionnaire (ACQ) score. In some embodiments, administration of the anti-LIGHT antibody increases the subject's Standardized Asthma Quality of Life Questionnaire score for 12 years and older (AQLQ(S)+12) score. In some embodiments, administration of the anti-LIGHT antibody decreases the subject's Asthma Symptom Diary score. In some embodiments, administration of the anti-LIGHT antibody improves the subject's European Quality of Life-5 Dimension 5 level Questionnaire score. In some embodiments, administration of the anti-LIGHT antibody improves the subject's Patient Global Impression of Change/Severity score. In some embodiments, administration of the anti-LIGHT antibody improves the subject's Clinician Global Impression of Improvement/Severity score. In some embodiments, administration of the anti-LIGHT antibody reduces the subject's incidence of SABA use. In some embodiments, the method further comprises assaying free LIGHT prior to, during, or after administration of the anti-LIGHT antibody. In some embodiments, the method further comprises assaying total LIGHT prior to, during, or after administration of the anti-LIGHT antibody. In some embodiments, the method further comprises assaying DcR3 prior to, during, or after administration of the anti-LIGHT antibody. In some embodiments, the subject has elevated free LIGHT. In some embodiments, administration of the anti-LIGHT antibody reduces serum free LIGHT in the subject.

In some embodiments, a method of treating a subject having non-eosinophilic asthma (NEA) is provided comprising administering an anti-LIGHT antibody to a subject in need thereof. In some embodiments, the subject has poorly controlled asthma as determined by an Asthma Control Questionnaire (ACQ) score ≥1.5. In some embodiments, the subject has poorly controlled asthma on a long-acting beta-agonist (LABA), optionally wherein the LABA is salmeterol, and on an inhaled corticosteroid (ICS), optionally wherein the ICS is fluticasone. In some embodiments, the subject has experienced exacerbation of asthma within 25 months prior to administration of a first dose of the anti-LIGHT antibody. In some embodiments, the subject has experienced exacerbation of asthma within 24 months prior to a screening visit. In some embodiments, the subject's blood eosinophil count <300 cells/μL. In some embodiments, the subject's blood eosinophil count <250 cells/μL. In some embodiments, the subject's blood eosinophil count <150 cells/μL. In some embodiments, the subject's blood eosinophil count is between 25 cells/μL and 325 cells/μL. In some embodiments, the subject's blood eosinophil count is between 25 cells/μL and 300 cells/μL. In some embodiments, the subject's blood eosinophil count is between 25 cells/μL and 250 cells/μL. In some embodiments, the subject's blood eosinophil count is between 25 cells/μL and 150 cells/μL. In some embodiments, the subject's blood eosinophil count is between 50 cells/μL and 325 cells/μL. In some embodiments, the subject's blood eosinophil count is between 50 cells/μL and 300 cells/μL. In some embodiments, the subject's blood eosinophil count is between 50 cells/μL and 250 cells/μL. In some embodiments, the subject's blood eosinophil count is between 50 cells/μL and 150 cells/μL. In some embodiments, the subject's blood eosinophil count is between 75 cells/μL and 325 cells/μL. In some embodiments, the subject's blood eosinophil count is between 75 cells/μL and 300 cells/μL. In some embodiments, the subject's blood eosinophil count is between 75 cells/μL and 250 cells/μL. In some embodiments, the subject's blood eosinophil count is between 75 cells/μL and 150 cells/μL. In some embodiments, the subject's blood eosinophil count is between 100 cells/μL and 200 cells/μL. In some embodiments, the subject's blood eosinophil count is between 150 cells/μL and 250 cells/μL. In some embodiments, the subject's blood eosinophil count is between 100 cells/μL and 150 cells/μL. In some embodiments, the subject is on a LABA at the time of administration of a first dose of the anti-LIGHT antibody, and the LABA is discontinued at about day 14 following administration of the first dose of the anti-LIGHT antibody. In some embodiments, the subject is on an ICS at the time of administration of a first dose of the anti-LIGHT antibody, and the ICS is reduced by 50% at about day 28 following administration of the first dose of the anti-LIGHT antibody. In some embodiments, the ICS is discontinued at about day 42 following the first administration of the anti-LIGHT antibody. In some embodiments, the anti-LIGHT antibody is administered at a dose of 3-12 mg/kg. In some embodiments, the anti-LIGHT antibody is administered at a dose of 3-11 mg/kg. In some embodiments, the anti-LIGHT antibody is administered at a dose of 3-10 mg/kg. In some embodiments, the anti-LIGHT antibody is administered at a dose of 3-9 mg/kg. In some embodiments, the anti-LIGHT antibody is administered at a dose of 3-8 mg/kg. In some embodiments, the anti-LIGHT antibody is administered at a dose of 3-7 mg/kg. In some embodiments, the anti-LIGHT antibody is administered at a dose of 3-6 mg/kg. In some embodiments, the anti-LIGHT antibody is administered at a dose of 3-5 mg/kg. In some embodiments, the anti-LIGHT antibody is administered at a dose of 3-4 mg/kg. In some embodiments, the anti-LIGHT antibody is administered at a dose of 6 mg/kg. In some embodiments, the anti-LIGHT antibody is administered at a dose of 7 mg/kg. In some embodiments, the anti-LIGHT antibody is administered at a dose of 8 mg/kg. In some embodiments, the anti-LIGHT antibody is administered at a dose of 100-1000 mg. In some embodiments, the anti-LIGHT antibody is administered at a dose of 100-900 mg. In some embodiments, the anti-LIGHT antibody is administered at a dose of 100-800 mg. In some embodiments, the anti-LIGHT antibody is administered at a dose of 100-700 mg. In some embodiments, the anti-LIGHT antibody is administered at a dose of 100-600 mg. In some embodiments, the anti-LIGHT antibody is administered at a dose of 100-500 mg. In some embodiments, the anti-LIGHT antibody is administered at a dose of 100-400 mg. In some embodiments, the anti-LIGHT antibody is administered at a dose of 100-300 mg. In some embodiments, the anti-LIGHT antibody is administered at a dose of 100-200 mg. In some embodiments, the anti-LIGHT antibody is administered at a dose of about 600 mg. In some embodiments, the anti-LIGHT antibody is administered at a dose of 600 mg. In some embodiments, the anti-LIGHT antibody is administered about every 14 days. In some embodiments, the anti-LIGHT antibody is administered about every 21 days. In some embodiments, the anti-LIGHT antibody is administered about every 28 days. In some embodiments, the anti-LIGHT antibody is administered about every 35 days. In some embodiments, the anti-LIGHT antibody is administered about every 42 days. In some embodiments, the anti-LIGHT antibody is administered about every 49 days. In some embodiments, the anti-LIGHT antibody is administered about every 56 days. In some embodiments, the anti-LIGHT antibody is administered every four weeks. In some embodiments, the anti-LIGHT antibody is administered monthly. In some embodiments, the anti-LIGHT antibody is administered at a dose of 8 mg/kg every four weeks. In some embodiments, the anti-LIGHT antibody is administered at a dose of 8 mg/kg monthly. In some embodiments, the anti-LIGHT antibody is administered at a dose of 6 mg/kg every four weeks. In some embodiments, the anti-LIGHT antibody is administered at a dose of 6 mg/kg monthly. In some embodiments, the anti-LIGHT antibody is administered at a dose of 600 mg every 28 days. In some embodiments, the anti-LIGHT antibody is administered at a dose of 600 mg monthly. In some embodiments, the anti-LIGHT antibody is administered for twelve weeks. In some embodiments, at least three doses of the anti-LIGHT antibody are administered. In some embodiments, the anti-LIGHT antibody is administered subcutaneously. In some embodiments, the anti-LIGHT antibody is administered intravenously. In some embodiments, the subject is human. In some embodiments, the subject is an adult. In some embodiments, the subject is a pediatric subject. In some embodiments, administration of the anti-LIGHT antibody increases the subject's time to exacerbation of asthma. In some embodiments, administration of the anti-LIGHT antibody increases the subject's time to an asthma related event. In some embodiments, administration of the anti-LIGHT antibody decreases the proportion of subjects in a population of subjects with exacerbation of asthma. In some embodiments, administration of the anti-LIGHT antibody decreases the proportion of subjects in a population of subjects with an asthma-related event. In some embodiments, the asthma related event is ≥6 additional reliever puffs of short-acting beta agonist (SABA) compared to baseline in a 24-hour period on 2 consecutive days, wherein baseline SABA use is determined by the average use in the 7 days preceding administration of a first dose of the anti-LIGHT antibody. In some embodiments, the asthma related event is an asthma exacerbation requiring the use of systemic corticosteroids (tablets, suspension, or injection) for at least 3 days. In some embodiments, the asthma related event is a hospitalization or emergency room visit because of an asthma exacerbation. In some embodiments, the asthma related event is increase in ICS dose ≥4 times than the dose at baseline, wherein baseline ICS dose is defined as the dosage the subject received during the 30 days leading up to administration of a first dose of the anti-LIGHT antibody. In some embodiments, the asthma related event is a decrease in peak flow of 30% or more (compared to baseline) on 2 consecutive days of treatment, wherein baseline peak flow is determined by the average of measurements in the 7 days preceding administration of a first dose of the anti-LIGHT antibody. In some embodiments, administration of the anti-LIGHT antibody increases the subject's forced expiratory volume in 1 second (FEV₁). In some embodiments, administration of the anti-LIGHT antibody decreases the subject's fractional exhaled nitric oxide (FeNO). In some embodiments, administration of the anti-LIGHT antibody decreases the subject's asthma control questionnaire (ACQ) score. In some embodiments, administration of the anti-LIGHT antibody increases the subject's Standardized Asthma Quality of Life Questionnaire score for 12 years and older (AQLQ(S)+12) score. In some embodiments, administration of the anti-LIGHT antibody decreases the subject's Asthma Symptom Diary score. In some embodiments, administration of the anti-LIGHT antibody improves the subject's European Quality of Life-5 Dimension 5 level Questionnaire score. In some embodiments, administration of the anti-LIGHT antibody improves the subject's Patient Global Impression of Change/Severity score. In some embodiments, administration of the anti-LIGHT antibody improves the subject's Clinician Global Impression of Improvement/Severity score. In some embodiments, administration of the anti-LIGHT antibody reduces the subject's incidence of SABA use. In some embodiments, the method further comprises assaying free LIGHT prior to, during, or after administration of the anti-LIGHT antibody. In some embodiments, the method further comprises assaying total LIGHT prior to, during, or after administration of the anti-LIGHT antibody. In some embodiments, the method further comprises assaying DcR3 prior to, during, or after administration of the anti-LIGHT antibody. In some embodiments, the subject has elevated free LIGHT. In some embodiments, administration of the anti-LIGHT antibody reduces serum free LIGHT in the subject.

### Anti-LIGHT Antibodies

In some embodiments, the anti-LIGHT antibody useful for therapeutic purposes may comprise the CDR sequences of the E1, E13, E63, F19, or F23 antibodies, which are provided in WO 2008/027338 and U.S. Pat. No. 8,058,402 B2, U.S. Pat. No. 8,461,307 B2, and U.S. Pat. No. 8,974,787 B2, each of which is incorporated herein by reference.

In some embodiments, the anti-LIGHT antibody comprises a heavy chain comprising three CDR sequences comprising each of SEQ ID NOs: 2, 3, and 4. In some embodiments, the anti-LIGHT antibody comprises a light chain comprising three CDR sequences comprising each of SEQ ID NOs: 5, 6, and 7. In some embodiments, the antibody comprises a heavy chain and a light chain, wherein the heavy chain comprises three CDR sequences comprising each of SEQ ID NOs: 2, 3, and 4, and wherein the light chain comprises three CDR sequences comprising each of SEQ ID NOs: 5, 6, and 7.

In some embodiments, the anti-LIGHT antibody comprises a heavy chain variable region sequence comprising SEQ ID NO: 84 or a sequence having at least 85%, at least 90%, at least 95%, at least 98%, or at least 99% identity to SEQ ID NO: 84. In some embodiments, the anti-LIGHT antibody comprises a light chain variable region sequence comprising SEQ ID NO: 85 or a sequence having at least 85%, at least 90%, at least 95%, at least 98%, or at least 99% identical to SEQ ID NO: 85. In some embodiments, the anti-LIGHT antibody comprises a heavy chain variable region sequence comprising SEQ ID NO: 84 or a sequence having at least 85%, at least 90%, at least 95%, at least 98%, or at least 99% identity to SEQ ID NO: 84, and a light chain variable region sequence comprising SEQ ID NO: 85 or a sequence having at least 85%, at least 90%, at least 95%, at least 98%, or at least 99% identical to SEQ ID NO: 85.

In some embodiments, the anti-LIGHT antibody comprises a heavy chain sequence comprising SEQ ID NO: 8 or a sequence having at least 85%, at least 90%, at least 95%, at least 98%, or at least 99% identity to SEQ ID NO:8. In some embodiments, the anti-LIGHT antibody comprises a light chain sequence comprising SEQ ID NO: 9 or a sequence having at least 85%, at least 90%, at least 95%, at least 98%, or at least 99% identical to SEQ ID NO: 9. In some embodiments, the anti-LIGHT antibody comprises both a heavy chain comprising SEQ ID NO: 8 or a sequence that is at least 85%, at least 90%, at least 95%, at least 98%, or at least 99% identical to SEQ ID NO:8 and a light chain comprising SEQ ID NO: 9 or a sequence that is at least 85%, at least 90%, at least 95%, at least 98%, or at least 99% identical to SEQ ID NO:9.

In some embodiments, the anti-LIGHT antibody comprises a heavy chain comprising three CDR sequences comprising each of SEQ ID NOs: 10, 11, and 12. In some embodiments, the anti-LIGHT antibody comprises a light chain comprising three CDR sequences comprising each of SEQ ID NOs: 13, 14, and 15. In some embodiments, the antibody comprises a heavy chain and a light chain, wherein the heavy chain comprises three CDR sequences comprising each of SEQ ID NOs: 10, 11, and 12, and wherein the light chain comprises three CDR sequences comprising each of SEQ ID NOs: 13, 14, and 15.

In some embodiments, the anti-LIGHT antibody comprises a heavy chain comprising three CDR sequences comprising each of SEQ ID NOs: 16, 17, and 18. In some embodiments, the anti-LIGHT antibody comprises a light chain comprising three CDR sequences comprising each of SEQ ID NOs: 19, 20, and 21. In some embodiments, the antibody comprises a heavy chain and a light chain, wherein the heavy chain comprises three CDR sequences comprising each of SEQ ID NOs: 16, 17, and 18, and wherein the light chain comprises three CDR sequences comprising each of SEQ ID NOs: 19, 20, and 21.

In some embodiments, the anti-LIGHT antibody comprises a heavy chain comprising three CDR sequences comprising each of SEQ ID NOs: 22, 23, and 24. In some embodiments, the anti-LIGHT antibody comprises a light chain comprising three CDR sequences comprising each of SEQ ID NOs: 25, 26, and 27. In some embodiments, the antibody comprises a heavy chain and a light chain, wherein the heavy chain comprises three CDR sequences comprising each of SEQ ID NOs: 22, 23, and 24, and the light chain comprises three CDR sequences comprising each of SEQ ID NOs: 25, 26, and 27.

In some embodiments, the anti-LIGHT antibody comprises a heavy chain comprising three CDR sequences comprising each of SEQ ID NOs: 28, 29, and 30. In some embodiments, the anti-LIGHT antibody comprises a light chain comprising three CDR sequences comprising each of SEQ ID NOs: 31, 32, and 33. In some embodiments, the antibody comprises a heavy chain and a light chain, wherein the heavy chain comprises three CDR sequences comprising each of SEQ ID NOs: 28, 29, and 30, and the light chain comprises three CDR sequences comprising each of SEQ ID NOs: 31, 32, and 33.

In some embodiments, the anti-LIGHT antibody comprises a heavy chain comprising three CDR sequences comprising each of SEQ ID NOs: 34, 35, and 36. In some embodiments, the anti-LIGHT antibody comprises a light chain comprising three CDR sequences comprising each of SEQ ID NOs: 37, 38, and 39. In some embodiments, the antibody comprises a heavy chain and a light chain, wherein the heavy chain comprises three CDR sequences comprising each of SEQ ID NOs: 34, 35, and 36, and the light chain comprises three CDR sequences comprising each of SEQ ID NOs: 37, 38, and 39.

In some embodiments, the anti-LIGHT antibody comprises a heavy chain comprising three CDR sequences comprising each of SEQ ID NOs: 40, 41, and 42. In some embodiments, the anti-LIGHT antibody comprises a light chain comprising three CDR sequences comprising each of SEQ ID NOs: 43, 44, and 45. In some embodiments, the antibody comprises a heavy chain and a light chain, wherein the heavy chain comprises three CDR sequences comprising each of SEQ ID NOs: 40, 41, and 42, and the light chain comprises three CDR sequences comprising each of SEQ ID NOs: 43, 44, and 45.

In some embodiments, the anti-LIGHT antibody comprises a heavy chain comprising three CDR sequences comprising each of SEQ ID NOs: 46, 47, and 48. In some embodiments, the anti-LIGHT antibody comprises a light chain comprising three CDR sequences comprising each of SEQ ID NOs: 49, 50, and 51. In some embodiments, the antibody comprises a heavy chain and a light chain, wherein the heavy chain comprises three CDR sequences comprising each of SEQ ID NOs: 46, 47, and 48, and the light chain comprises three CDR sequences comprises each of SEQ ID NOs: 49, 50, and 51.

In some embodiments, the anti-LIGHT antibody may comprise the CDR sequences of the antibodies which are described in US2013/0323240 and U.S. Pat. No. 8,524,869 B2, which are incorporated herein by reference. For example, in some embodiments, the anti-LIGHT antibody comprises a heavy chain comprising three CDR sequences comprising each of SEQ ID NOs: 52, 53, and 54, respectively. In some embodiments, the anti-LIGHT antibody comprises a light chain comprising three CDR sequences comprising each of SEQ ID NOs: 55, 56, and 57, respectively. In some embodiments, the antibody comprises a heavy chain and a light chain, the heavy chain comprising three CDR sequences comprising each of SEQ ID NOs: 52, 53, and 54, respectively, and the light chain comprising three CDR sequences comprising each of SEQ ID NOs: 55, 56, and 57, respectively.

In some embodiments, the anti-LIGHT antibody comprises a heavy chain variable region sequence comprising SEQ ID NO:58 or that is at least 85%, at least 90%, at least 95%, at least 98%, or at least 99% identical to SEQ ID NO:58. In some embodiments, the anti-LIGHT antibody comprises a light chain variable region sequence comprising SEQ ID NO:59 or that is at least 85%, at least 90%, at least 95%, at least 98%, or at least 99% identical to SEQ ID NO:59. In some embodiments, the anti-LIGHT antibody comprises a heavy chain comprising SEQ ID NO: 58 or that is at least 85%, at least 90%, at least 95%, at least 98%, or at least 99% identical to SEQ ID NO:58. In some embodiments, the anti-LIGHT antibody comprises a light chain comprising SEQ ID NO:59 or that is at least 85%, at least 90%, at least 95%, at least 98%, or at least 99% identical to SEQ ID NO:59. In some embodiments, the anti-LIGHT antibody comprises both a heavy chain comprising SEQ ID NO:58 or that is at least 85%, at least 90%, at least 95%, at least 98%, or at least 99% identical to SEQ ID NO:58 and a light chain comprising SEQ ID NO:59 or that is at least 85%, at least 90%, at least 95%, at least 98%, or at least 99% identical to SEQ ID NO:59.

In some embodiments, the anti-LIGHT antibody may comprise a heavy chain and a light chain together comprising one of the following sets of CDR-H1, CDR-H2, CDR-H3, CDR-L1, CDR-L2, and CDR-L3 sequences described in the sequence listing from US2013/0323240: SEQ ID NOs: 18, 19, 20 and SEQ ID NOs: 38, 41, 42 of US2013/0323240; SEQ ID NOs: 18, 19, 21 and SEQ ID NOs: 39, 41, 42 of US2013/0323240; SEQ ID NOs: 18, 19, 22 and SEQ ID NOs: 40, 41, 42 of US2013/0323240; SEQ ID NOs: 23, 24, 25 and SEQ ID NOs: 43, 44, 45 of US2013/0323240; SEQ ID NOs: 26, 27, 28 and SEQ ID NOs: 46, 47, 48 of US2013/0323240; SEQ ID NOs: 29, 30, 31 and SEQ ID NOs: 49, 50, 51 of US2013/0323240; SEQ ID NOs: 32, 33, 34 and SEQ ID NOs: 52, 53, 54 of US2013/0323240; and SEQ ID NOs: 35, 36, 37 and SEQ ID NOs: 55, 50, 51 of US2013/0323240.

In some embodiments, the anti-LIGHT antibody comprises the CDR sequences of the 18E04, 98C07, 1C02, 1C06, 13H04, 31A10, 98C07, 42A02, 29C02, 14B09, 117C06, 114F05, and 62C01 antibodies described in WO 2015/107331, which is also incorporated by reference herein.

For example, in some embodiments, the anti-LIGHT antibody comprises a heavy chain comprising three CDR sequences comprising each of SEQ ID NOs: 60, 61, and 62, respectively. In some embodiments, the anti-LIGHT antibody comprises a light chain comprising three CDR sequences comprising each of SEQ ID NOs: 63, 64, and 65, respectively. In some embodiments, the antibody comprises a heavy chain and a light chain, the heavy chain comprising three CDR sequences comprising each of SEQ ID NOs: 60, 61, and 62, respectively, and the light chain comprising three CDR sequences comprising each of SEQ ID NOs: 63, 64, and 65, respectively.

In some embodiments, the anti-LIGHT antibody comprises a heavy chain comprising three CDR sequences comprising each of SEQ ID NOs: 66, 67, and 68, respectively. In some embodiments, the anti-LIGHT antibody comprises a light chain comprising three CDR sequences comprising each of SEQ ID NOs: 69, 70, and 71, respectively. In some embodiments, the antibody comprises a heavy chain and a light chain, the heavy chain comprising three CDR sequences comprising each of SEQ ID NOs: 66, 67, and 68, respectively, and the light chain comprising three CDR sequences comprising each of SEQ ID NOs: 69, 70, and 71, respectively.

In some embodiments, the anti-LIGHT antibody comprises a heavy chain comprising three CDR sequences comprising each of SEQ ID NOs: 72, 73, and 74, respectively. In some embodiments, the anti-LIGHT antibody comprises a light chain comprising three CDR sequences comprising each of SEQ ID NOs: 75, 76, and 77, respectively. In some embodiments, the antibody comprises a heavy chain and a light chain, the heavy chain comprising three CDR sequences comprising each of SEQ ID NOs: 72, 73, and 74, respectively, and the light chain comprising three CDR sequences comprising each of SEQ ID NOs: 75, 76, and 77, respectively.

In some embodiments, the anti-LIGHT antibody comprises a heavy chain comprising three CDR sequences comprising each of SEQ ID NOs: 78, 79, and 80, respectively. In some embodiments, the anti-LIGHT antibody comprises a light chain comprising three CDR sequences comprising each of SEQ ID NOs: 81, 82, and 83, respectively. In some embodiments, the antibody comprises a heavy chain and a light chain, the heavy chain comprising three CDR sequences comprising each of SEQ ID NOs: 78, 79, and 80, respectively, and the light chain comprising three CDR sequences comprising each of SEQ ID NOs: 81, 82, and 83, respectively.

### Free LIGHT Detection Assays

Most currently available assays only measure total LIGHT, which includes LIGHT bound to its receptors, including DcR3. Total LIGHT may not provide as accurate of a picture of the levels of LIGHT causing disease, which may be free, unbound LIGHT. Thus, it may be desirable to use an assay that measures free LIGHT alone when the methods include detection of free LIGHT. Assays that measure free LIGHT are described in WO2021/202649, which is incorporated herein by reference in its entirety, and in particular, for its description of free LIGHT assays.

### EXAMPLES

The following examples are provided to illustrate certain disclosed embodiments and are not to be construed as limiting the scope of this disclosure in any way. In the Examples discussed below, “Antibody A” refers to an anti-LIGHT antibody, wherein the anti-LIGHT antibody comprises the following six CDRs: a heavy chain CDR1 having an amino acid sequence of SEQ ID NO: 2; a heavy chain CDR2 having an amino acid sequence of SEQ ID NO: 3; a heavy chain CDR3 having an amino acid sequence of SEQ ID NO: 4; a light chain CDR1 having an amino acid sequence of SEQ ID NO: 5; a light chain CDR2 having an amino acid sequence of SEQ ID NO: 6; and a light chain CDR3 having an amino acid sequence of SEQ ID NO: 7. In some embodiments Antibody A has a variable heavy chain (VH) having an amino acid sequence of SEQ ID NO: 84 and a variable light chain (VL) having an amino acid sequence of SEQ ID NO: 85. In some embodiments Antibody A has a heavy chain having an amino acid sequence of SEQ ID NO: 8 and a light chain having an amino acid sequence of SEQ ID NO: 9.

### Example 1—A Phase 2, Randomized, Double-Blind, Placebo-Controlled, Parallel Group Study to Evaluate the Safety and Efficacy of Antibody A for the Treatment of Poorly Controlled Non-Eosinophilic Asthma

### Example 1.1—Study Objectives and Endpoints

The primary objective of the study is to assess the ability of Antibody A to improve asthma control in subjects with poorly controlled NEA based on the proportion of patients who experience asthma related events.

The secondary objectives of the study are: to assess the safety and tolerability of Antibody A; to evaluate biomarkers of pharmacodynamic (PD) activity and mechanism of action of Antibody A; and to evaluate the immunogenicity of Antibody A.

The exploratory objectives of the study are: to assess the ability of Antibody A to improve asthma control in subjects with poorly controlled NEA who also have <150 eosinophils/μL; to assess sputum soluble LIGHT levels and gene expression in a select subset of subjects; to evaluate inflammatory proteins including but not limited to: interleukin (IL)-6, interferon gamma (IFN-y) and tumor necrosis factor alpha (TNF-α); and to evaluate the PK/PD relationship.

The primary endpoint of the study is the proportion of patients who experience any of the following asthma related events: ≥6 additional reliever puffs of SABA (compared to baseline) in a 24-hour period on 2 consecutive days (Baseline SABA use will be determined by the average use in the 7 days preceding Visit 2); increase in ICS dose ≥4 times than the dose at baseline (baseline ICS dose is defined as the dosage the subject received during the 30-day run-in period); a decrease in peak flow of 30% or more (compared to baseline) on 2 consecutive days of treatment (baseline peak flow will be determined by the average of measurements in the 7 days preceding Visit 2); an asthma exacerbation requiring the use of systemic corticosteroids (tablets, suspension, or injection) for at least 3 days; or a hospitalization or emergency room visit because of an asthma exacerbation.

The secondary endpoints of the study are: change from baseline in forced expiratory volume in 1 second (FEV₁) at Weeks 2, 4, 6, 8, 12, and 14; time to asthma related event; change from baseline in fractional exhaled nitric oxide (FeNO) at Weeks 2, 4, 6, 8, 12, and 14; change from baseline in Asthma Control Questionnaire (ACQ) at Weeks 2, 4, 6, 8, 12, and 14; change from baseline in Standardized Asthma Quality of Life Questionnaire for 12 years and older (AQLQ(S)+12) at Weeks 2, 4, 6, 8, 12, and 14; change from baseline in Asthma Symptom Diary Score at Weeks 2, 4, 6, 8, 12, and 14; change from baseline in European Quality of Life-5 Dimension 5 level Questionnaire at Weeks 2, 4, 6, 8, 12, and 14; change from baseline in Patient Global Impression of Change/Severity at Weeks 2, 4, 6, 8, 12, and 14; change from baseline in Clinician Global Impression of Improvement/Severity at Weeks 2, 4, 6, 8, 12, and 14; incidence of short-acting beta agonist (SABA) use at Weeks 2, 4, 6, 8, 12, and 14; change from baseline in serum soluble LIGHT levels at Weeks 2, 4, 6, 8, 12, and 14; incidence of adverse events (AEs), and changes from baseline in clinical laboratory tests, vital signs measurements, and physical examinations at Weeks 2, 4, 6, 8, 12, and 14; and incidence of ADAs at Weeks 2, 4, 6, 8, 12, and 14.

The exploratory endpoints of the study are: occurrence of asthma related events in subjects with <150 eosinophils/μL; change from baseline in sputum soluble LIGHT and gene expression (select subset of site/subjects only); change from baseline in inflammatory proteins including but not limited to: IL-6, IFN-γ, and tumor necrosis factor alpha (TNF-α); and PK/PD assessments.

### Example 1.2—Study Design

This is a randomized, double-blind, placebo-controlled, Phase 2 study to evaluate the safety and efficacy of Antibody A for the treatment of poorly controlled NEA. Upon confirmation of eligibility, subjects will complete a 30-day, salmeterol and fluticasone stable dose, run-in prior to randomization. At Baseline subjects will be randomized to receive either three doses of Antibody A at 600 mg (Days 0, 28 and 56) or they will receive 3 doses of placebo at the same time points. Subjects will then begin a withdrawal of salmeterol and fluticasone therapies. Subjects will discontinue salmeterol at Day 14 and then taper fluticasone use to 50% dose at Day 28. Fluticasone use will be completely discontinued at Day 42. Subjects will be followed until Day 98.

An adequate number of subjects will be enrolled to ensure approximately 80 subjects complete the study.

All subjects will undergo efficacy, safety, PK/PD, and immunogenicity assessments. The efficacy of Antibody A will be determined by the occurrence of an asthma related event (primary endpoint), time to asthma related event, and change from baseline in the following parameters: FEV₁; FeNO; ACQ; AQLQ(S)+12); Asthma Symptom Diary Score; European Quality of Life-5 Dimension 5 level Questionnaire; Patient Global Impression of Change/Severity (PGI-C/S); Clinician Global Impression of Improvement/Severity (CGI-I/S); and Incidence of short-acting beta agonist (SABA) use. All subjects will be monitored for AEs and will undergo physical exams, vital signs, and routine safety laboratory tests. The PK/PD of Antibody A will be determined by measuring the levels of Antibody A and LIGHT in addition to other biomarkers of NEA. Finally, the immunogenicity of Antibody A will be determined by measuring ADA levels.

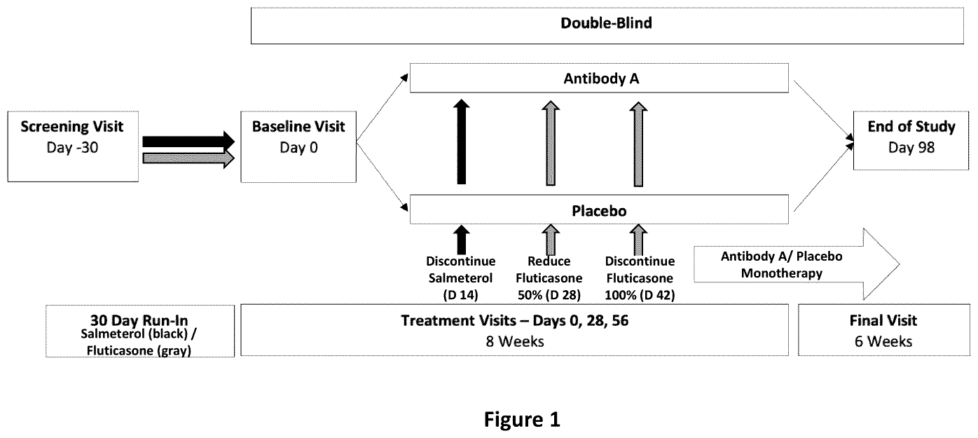

The study schema is displayed in FIG. 1.

This study will assess safety and efficacy of Antibody A in subjects with poorly controlled non-eosinophilic asthma.

Subjects will be evaluated for enrollment at the Screening visit. If considered eligible for enrollment, subjects will undergo a 30-day, stable dose, run-in period with a LABA (salmeterol) and an ICS (fluticasone). Documentation of eligibility on all Inclusion and Exclusion Criteria is required before a subject can enter the study 30-day run-in period prior to Visit 2 (Randomization) when the subject is switched to stand alone Salmeterol and Fluticasone. Confirmation of eligibility should take place within a reasonable amount of time after the Screening Visit. If confirmation of eligibility is not available within 7 days from the date of Screening, the study sponsor should provide approval for the subject to continue.

During the 30-day run-in, a telephone visit (Visit 1a) should occur 7 days prior to Baseline, at Day-7 (−2 day window). During this telephone visit, the subject should be reminded to begin daily peak flow measurements as well as completion of the daily diary to capture ASD, SABA use and daily peak flow values.

After the run-in period, subjects will be randomized at the Baseline Visit (Day 0) to either Antibody A administered at 600 mg or placebo. Subjects will receive Antibody A or placebo at Visit 2 (Day 0), Visit 5 (Day 28) and Visit 7 (Day 56). Over the course of the first 42 days after randomization subjects will taper or discontinue use of products administered during their run-in period. Salmeterol will be discontinued at Visit 4 (Day 14), fluticasone use will be tapered to 50% dose at Visit 5 (Day 28) and fluticasone will be discontinued completely at Visit 6 (Day 42). All subjects will proceed on Antibody A or placebo monotherapy until Visit 9 (Day 98), which is also the end of study visit. Efficacy will be assessed from Baseline Visit (Day 0) through Visit 9 (Day 98) per the schedule outlined in schedule of assessments (Table1).

The run-in period after Screening and before Baseline must be completed with a stand-alone LABA (salmeterol) and an ICS (fluticasone). Principal investigators will ensure subjects are transitioned to these products at the Screening visit if the subject is not already on these products.

If a subject must discontinue the study prior to Visit 9 (Day 98) for any reason, the subject will be required to complete an Early Termination Visit (Day 98 procedures).

The study will take place at approximately 25 study sites in the US.

Subjects diagnosed with NEA (<300 eosinophils/μL) (measured in blood) will be randomized into the study. An adequate number of subjects will be enrolled to ensure approximately 80 subjects complete the study.

Inclusion Criteria Subjects must fulfill the following requirements to be randomized into the study:
1. Subject is ≥18 years of age at the time of informed consent.
2. Documented non-eosinophilic asthma diagnosis (<300 eosinophils/μL in blood).
3. Symptoms consistent with a diagnosis of asthma that is poorly controlled as determined by an ACQ score ≥1.5.
4. Poorly controlled asthma despite the use of a LABA and ICS for at least 3 consecutive months immediately prior to the Screening Visit.
5. Subjects must have had at least one asthma exacerbation in the last 24 months prior to Screening.
6. Non-pregnant, non-lactating female subjects of childbearing potential who are heterosexually active and non-sterile male subjects with female sexual partners of childbearing potential agree to use a highly effective method of contraception during the treatment period and for 28 days following the last dose of study medication. A highly effective method of birth control is defined as one that results in a low failure rate (i.e., <1% per year) when used consistently and correctly, such as oral/injectable/inserted/implanted/transdermal contraceptives, condom with diaphragm, condom with spermicide, diaphragm with spermicide, intrauterine hormone-releasing system, or intrauterine device (IUD), or sexual abstinence. Contraception is not required where at least 6 weeks have passed since sterilization, defined as females having undergone one of the following surgeries: hysterectomy, bilateral tubal ligation or occlusion, bilateral oophorectomy, or bilateral salpingectomy; and males who are vasectomized. Contraception is not required where females are postmenopausal (defined as 12 consecutive months of spontaneous amenorrhea and age ≥51 years). Females of childbearing potential must have a negative pregnancy test as part of the screening assessment.
7. Subject can understand and provide informed consent to participate in this study.

### Exclusion Criteria

The presence of any of the following criteria excludes a subject from the study:
1. Pulmonary disease other than asthma.
2. Currently on biologic therapy. Previous biologic therapy is permitted with adequate washout (12 weeks or 5 half-lives, whichever is longer).
3. Subjects who: are current smokers; have a history of smoking >/=10 pack years; have a history of inhaled cannabis >4 days/week for the most recent 3 months prior to screening; or use e-cigarettes or vaping devices >4 days/week for the most recent 3 months prior to screening.
4. Current suspected drug or alcohol abuse.
5. Use of systemic immunosuppressants within the last 6 months.
6. Use of systemic corticosteroids within 6 weeks prior to Screening or use of antibiotics within 4 weeks prior to Screening.
7. Subjects that are pregnant or breastfeeding.
8. Subject has a history of neoplasia except for a curatively treated non-melanoma skin tumor or carcinoma of the cervix treated in situ without any indication of recurrence within the 10 years prior to Visit 1.
9. Subject has a chronic, active infection or another disease, which entails a tendency towards infection.
10. Subject has a chronic severe or uncontrolled medical disorder that might confound the results of safety assessments conducted in this study.
11. Subject has a history of unresolved latent tuberculosis.
12. Subject has alanine aminotransferase (ALT)/aspartate aminotransferase (AST)>5 upper limit of normal (ULN) and/or serum creatinine concentration >1.5 mg/dL.
13. Subject has hemoglobin≤10 g/dL, neutrophils≤1,500/μl, and/or platelets≤75,000/μl.
14. Subject has received a live vaccine within 12 weeks prior to Visit 2.
15. Subject has used an investigational product, including Emergency Use Authorization (EUA) vaccines, within 30 days of Visit 1.
16. Subject has known or suspected intolerance or hypersensitivity to the investigational product(s), closely related compounds, or any ingredients of the investigational product.
17. There is any concern on the part of the investigator regarding the subject's safety, compliance, or suitability with respect to his/her participation in the study.

### Screen Failures

Subjects who fail inclusion and/or exclusion criteria may be rescreened for the study with sponsor approval.

### Premature Subject Withdrawal

All subjects will be advised that they are free to withdraw from participation in this study at any time, for any reason, and without prejudice. Every reasonable attempt should be made by the investigator to keep subjects in the study; however, subjects must be withdrawn from the study if they withdraw consent to participate. Investigators must attempt to contact subjects who fail to attend scheduled visits by telephone or other means to exclude the possibility of an AE being the cause of withdrawal. Should this be the cause, the AE must be documented, reported, and followed.

Subjects can decline to continue receiving study drug at any time during the study. If this occurs, the investigator is to discuss with the subject the completion of the Early Termination Visit to occur 4 weeks (±3 days) post last dose. If the subject refuses this visit/procedures associated with this visit, data on concomitant medications and AEs will be collected if the subject agrees. Data on concomitant medications and AEs can be collected via a telephone call if the subject refuses an in-person visit.

Withdrawal of consent for a study means the subject does not wish to receive further protocol-required treatment or procedures, and the subject does not wish to or is unable to continue further study participation. Subject data up to withdrawal of consent will be included in the analysis of the study, and where permitted, publicly available data can be included after withdrawal of consent.

The Sponsor reserves the right to request the withdrawal of a subject due to protocol deviations or other reasons.

The investigator also has the right to withdraw subjects from the study at any time for any reason. If a subject is withdrawn before completing the study, the subject should be followed-up as instructed in the schedule of assessments (Table 1). The reason for withdrawal must be determined by the investigator and recorded in the subject's medical record and on the electronic case report form (eCRF) as well as the date of discontinuation. If a subject is withdrawn for more than 1 reason, each reason should be documented in the source document and the most clinically relevant reason should be entered on the eCRF.

Reasons for discontinuation include but are not limited to:
Lack of efficacy
Death
Protocol deviation
Physician decision
Sponsor request
Withdrawal by subject
Lost to follow-up.
Other (specify). For example, pregnancy.

TABLE 1
Schedule of Assessments

| | Procedure | | | | | | | | | | |
|---|---|---|---|---|---|---|---|---|---|---|---|
| | | 30-Day Run- | | | Double-Blind | | | | | | |
| | Screening | In Period | | Baseline | Treatment Period | | | | | | |
| | Visit | | | | | | | | | | |
| | Visit | | Visit | Visit | Visit | Visit | Visit | Visit | Visit | Visit | Visit |
| | 1 | | 1a | 2 | 3 | 4 | 5 | 6 | 7 | 8 | 9/ET² |
| | Assessment Week | | | | | | | | | | |
| | −4 | | −1 | 0 | 1 | 2 | 4 | 6 | 8 | 12 | 14 |
| | Assessment Day | | | | | | | | | | |
| | | −30 | | | | | | | No later | | |
| | Approximately −37¹ | Through −1 | −7 | 0 | 7 | 14 | 28 | 42 | than 56³ | 84 | 98 |
| | Visit Window⁴ | | | | | | | | | | |
| | | | | +3 | ±1 | ±1 | ±3 | ±3 | −3 | ±3 | ±3 |
| | NA | | −2 | days⁵ | day | day | days | days | days | days | days |
| Phone Visit | | | X⁶ | | | | | | | | |
| Informed consent | X | | | | | | | | | | |
| Inclusion/exclusion review | X | | | X | | | | | | | |
| Demographics | X | | | | | | | | | | |
| Medical history | X | | | | | | | | | | |
| Prior medications | X | X | X | | | | | | | | |
| Randomization | | | | X | | | | | | | |
| Concomitant medications | | | | X | X | X | X | X | X | X | X |
| Adverse events | X | X | X | X | X | X | X | X | X | X | X |
| Vital signs | X⁸ | | | X⁷ | | X | X⁷ | X | X⁷ | X | X |
| Physical examination | X | | | X | | X | X | X | X | X | X |
| 12-lead ECG | X | | | | | | | | | | |
| FEV1 | X | | | X | | X | X | X | X | X | X |
| FeNO | X | | | X | | X | X | X | X | X | X |
| Peak Flow Test (Daily)⁹ | | | X | X | X | X | X | X | X | X | X |
| ACQ | X | | | X | | X | X | X | X | X | X |
| AQLQ(S) + 12 | | | | X | | X | X | X | X | X | X |
| Asthma Symptom | | | X | X | X | X | X | X | X | X | X |
| Diary (2xDaily) | | | | | | | | | | | |
| Record SABA Use | | | X | X | X | X | X | X | X | X | X |
| (2xDaily) | | | | | | | | | | | |
| European Quality | | | | X | | X | X | X | X | X | X |
| of Life − 5 | | | | | | | | | | | |
| Dimension 5 level | | | | | | | | | | | |
| Questionnaire | | | | | | | | | | | |
| Patient Global Impression | | | | X | | X | X | X | X | X | X |
| of Change/Severity | | | | | | | | | | | |
| Clinician Global | | | | X | | X | X | X | X | X | X |
| Impression of | | | | | | | | | | | |
| Improvement/Severity | | | | | | | | | | | |
| ADA¹⁰ | | | | X | | X | X | X | X | X | X |
| Serum: soluble LIGHT | | | | X | | X | X | X | X | X | X |
| Inflammatory proteins | X | | | | | | X | | X | X | X |
| Immunophenotyping of | | | | X | | | | | | X | X¹⁶ |
| circulating immune cells | | | | | | | | | | | |
| Sputum: soluble LIGHT | | | | X | | | | | | X | |
| and gene expression¹¹ | | | | | | | | | | | |
| Plasma: Antibody A level | | | | X | X | X | X | X | X | X | |
| Clinical laboratory tests | X | | | X | | X | X | X | X | X | X |
| (hematology, clinical | | | | | | | | | | | |
| chemistries) | | | | | | | | | | | |
| Urinalysis | X | | | X | | X | | | X | X | X |
| Serum pregnancy test ¹² | X | | | | | | | | | X | X |
| Urine pregnancy test ¹² | | | | X | | | X | | X | | |
| Study drug administration | | | | X | | | X | | X | | |
| onsite ^{13, 14, 15} | | | | | | | | | | | |
| Salmeterol/Fluticasone | | X | X | | | | | | | | |
| Run-In | | | | | | | | | | | |
| Discontinue salmeterol | | | | | | X | | | | | |
| Taper/discontinue | | | | | | | X | X | | | |
| fluticasone | | | | | | | | | | | |

Abbreviations: ACQ = Asthma Control Questionnaire; ADA = anti-drug antibody; AQLQ(S) + 12 = Standardized Asthma Quality of Life Questionnaire for 12 years and older; ECG = electrocardiogram; ET = early termination; FeNO = fractional exhaled nitric oxide; FEV1 = forced expiratory volume in 1 second; LIGHT = Lymphotoxin like, exhibits Inducible expression, and competes with Herpes Virus Glycoprotein D for Herpesvirus Entry Mediator.
¹Documentation of eligibility on all Inclusion and Exclusion Criteria is required before a subject can enter the study 30-day run-in period prior to Visit 2 (Randomization) when the subject is switched to stand alone Salmeterol and Fluticasone. Confirmation of eligibility should take place within a reasonable amount of time after the Screening Visit. If confirmation of eligibility is not available within 7 days from the date of Screening, the study sponsor should provide approval for the subject to continue.
²If a subject discontinues from the study, the Visit 9/ET procedures will be performed 4 weeks post last dose (±3 days).
³A subject must be dosed with the Visit 7 (Week 8) dose no later than Day 56.
⁴After Visit 2 (Day 0), visits should be scheduled relative to Visit 2 (Day 0). Visit 7 (Week 8) must not be performed later than Day 56.
⁵Baseline visit to occur the day following completion of the 30 day run-in period with a window of an additional 3 days.
⁶Visit 1a is a telephone visit that should occur with 7 days remaining in the 30-Day Run-In (−2 Day window for Visit 1a). During this phone call, the subject should be reminded to begin daily peak flow testing, and daily completion of their diary to capture asthma symptoms, SABA use and daily peak flow values.
⁷At Visit 2 (Day 0), Visit 5 (Day 28), and Visit 7 (Day 56), pre- and post-dose vital signs will be measured: Pre-dose vital signs should be taken within 60 minutes before dosing. Post-dose vital signs should be taken at least 60 minutes after dosing, prior to discharge.
⁸Height will only be recorded at the Screening Visit.
⁹Subjects should record the best result of 3 attempts daily at home. Subjects should perform daily peak flow at approximately the same time each day between 10:00 AM and 2:00 PM. Subject should also bring peak flow meter to clinic visits. If peak flow has not been completed at home by the subject on the day of a clinic visit it should be completed at the clinic during the visit.
¹⁰In addition to ADA collection timepoints per the schedule of events, an ADA sample will be collected if an immunologically related adverse event is reported (e.g., a skin reaction, lupus-like syndrome, unexplained thrombocytopenia).
¹¹ Select subset of sites/subjects only.
¹² For females of childbearing potential. A subject is not considered to be of childbearing potential if at least 6 weeks have passed since sterilization, defined as females having undergone one of the following surgeries: hysterectomy, bilateral tubal ligation or occlusion, bilateral oophorectomy, or bilateral salpingectomy; and males who are vasectomized. Contraception is not required where females are postmenopausal (defined as 12 consecutive months of spontaneous amenorrhea and age ≥51 years).
¹³ On the day of Randomization when the first IP administration is required (Visit 2/Day 0), all visit procedures should be completed prior to IP administration. At the other IP administration Visit dates (Visit 5/Day 28 and Visit 7/Day 56), only the pharmacokinetic (PK), anti-drug antibody (ADA), Serum soluble LIGHT, inflammatory proteins and immunophenotyping samples must be collected prior to dosing. Other procedures can be performed as needed in the clinic on the visit day.
¹⁴ Subjects will be required to remain in the clinic for at least 60 minutes after study drug administration for adverse event monitoring.
¹⁵ Investigational product will be prepared by an unblinded pharmacist or appropriately qualified individual.
¹⁶Immunophenotyping only required if an Early Termination visit is completed. Immunophenotyping not required at Visit 9/Day 98.

### Example 1.3—Treatments

### Identification of Investigational Product, Dose and Mode of Administration

Antibody A is the investigational product that will be used in this study. Antibody A will be supplied in single-use vials containing 300 mg Antibody A (concentration 150 mg/mL). Placebo will be sourced locally and provided as volume-matched normal saline for injection. Antibody A or placebo will be administered by subcutaneous (SC) injection in the abdomen in a zone of 4 to 10 cm from the umbilicus, with the injection site rotated based on the number of syringes used. Antibody A or placebo will be administered at Visit 2 (Day 0), Visit 5 (Day 28), and Visit 7 (Day 56). Antibody A will be administered at a dose of 600 mg.

### Treatments Administered

Eligible subjects will receive 600 mg Antibody A or placebo subcutaneously (SC) on Visit 2 (Day 0), Visit 5 (Day 28), and Visit 7 (Day 56).

### Blinding and Unblinding Treatment Assignment

This is a double-blind study. All subjects, investigators, and study personnel involved in the conduct of the study will be blinded to treatment assignment except as outlined in study specific blind management plans.

Treatment unblinding is discouraged if knowledge of the treatment assignment will not materially change the planned management of a medical emergency. Unblinding is permitted in a medical emergency that requires immediate knowledge of the subject's treatment assignment. Whenever possible unblinding should be discussed with the medical monitor. For emergency unblinding, the investigator can unblind in the IXRS/eCRF. If the investigator is not able to discuss treatment unblinding with the medical monitor in advance of the unblinding, then they should notify the medical monitor as soon as possible about the unblinding incident without revealing the subject's treatment assignment. The investigator or designee must record the date and reason for study discontinuation on the appropriate eCRF for that subject. In all cases that are not emergencies, the investigator should discuss the event with the medical monitor prior to unblinding the subject's treatment assignment.

If the treatment assignment is unblinded for an individual subject, the investigator will be notified of that subject's treatment assignment without unblinding the treatment assignments for the remaining subjects in the study.

### Selection of Doses in the Study

In the Phase 1 first in human study, single dose Antibody A was absorbed with the median tₘₐₓ ranging from 5.0 to 8.5 days and eliminated with the mean terminal half-life ranging from 18 days to 27 days.

Preliminary pharmacokinetic data is now available from a study of Antibody A in patients with Crohn's Disease (clinicaltrials.gov identifier NCT03169894), with peak serum concentrations of Antibody A not achieved until at least study day 21, which is after the second dose administration (study day 14), confirming predicted accumulation with repeat dosing based on Phase 1 single dose pharmacokinetic data.

In this study, the Antibody A dosing schedule is once every 28 days (one 600 mg dose/month) versus every 14 days (one 300 mg dose twice/month) in the Crohn's Disease study. Based on the PK data and favorable safety profile of biweekly dosing up to 3 mg/kg in the Crohn's Disease study, the Antibody A dose in this study will be 600 mg once monthly for a total of 3 doses. This dosing regimen is expected to provide higher serum Antibody A concentrations prior to discontinuation of salmeterol at study day 14 with the 28-day dosing interval limiting the potential for accumulation with the 2 subsequent doses.

### Dose Adjustment Criteria

No dose adjustments are allowed.

### Non-Study Therapies, ICS, and LABA Use

All non-study therapies including but not limited to over the counter and non-pharmacological treatments from the time of Screening (Visit 1) and through the end of study must be recorded on the appropriate eCRF page. Record of ICS and LABA use should be captured for the three months immediately prior to screening.

### Prior Therapies

Prior therapies include all therapies received from the Screening Visit (Visit 1) to just prior to 1^{st} dose of study drug, with the exception of ICS and LABA which should also be recorded for the 3 months immediately prior to Screening (Visit 1). Prior therapy information must be recorded on the appropriate eCRF page.

### Concomitant Therapies

Concomitant therapies refer to all therapies taken on or after the first dose of study drug through the last visit. Concomitant therapy information must be recorded on the appropriate eCRF page.

### Permitted Therapies

Medications considered necessary for the subject's welfare, may be administered at the discretion of the investigator. The medical monitor should be contacted in the event the site is in a situation where further clarity is needed.

Acceptable methods of birth control considered to be highly effective (e.g., results in a low failure rate [i.e., <1% per year]) when used consistently and correctly, include oral/injectable/inserted/implanted/transdermal contraceptives, co study medication and study condom with diaphragm, condom with spermicide, diaphragm with spermicide, intrauterine hormone-releasing system or IUD, or sexual abstinence.

### Prohibited Therapies

During the study, new initiation of investigational compounds or concomitant treatment with other asthma therapy is not permitted. The use of systemic corticosteroids, biologics, immunosuppressants and inhaled anticholinergics is not permitted. Leukotriene inhibitors are not allowed to be added to a subject's treatment regimen after they are screened for the study. Subjects who are on leukotriene inhibitors as part of their prior therapy at Screening (Visit 1) may continue on leukotriene inhibitors during the course of study participation.

Treatment after End of Study

Subjects will be treated per standard clinical practice following completion of participation in the study.

### Example 1.4—Study Procedures

### Study Duration

Antibody A and placebo treatment will be administered at Visit 2 (Day 0), Visit 5 (Day 28), and Visit 7 (Day 56). A subject may not be dosed after Day 56. The duration of the treatment period is considered 84 days, with each dose of study drug considered to be 28 days of treatment. The overall study period is approximately 135 days, including Screening and the 30-day, stable dose, run-in period.

### Medical History Assessments

A medical and surgical history will be taken at Screening. All significant medical history findings that have been present or active within the 5 years prior to screening will be entered into the eCRF. Medical history findings that have not been present within the 5 years prior to screening will be recorded if deemed clinically relevant by the investigator to the conduct of the study.

Additionally, the subject's overall NEA history will be captured at Screening. This includes but is not limited to the approximate date of diagnosis and prior treatment including approximate start and stop dates.

### Efficacy Assessments

Efficacy response will be assessed by the procedures listed below at the time points mentioned in the schedule of assessments (Table 1).

### Asthma Related Events

The proportion of patients who experience any of the following asthma related events and time to the asthma related event will be determined:
≥6 additional reliever puffs of SABA (compared to baseline) in a 24-hour period on 2 consecutive days or,
   Baseline SABA use will be determined by the average use in the 7 days preceding Visit 2,
increase in ICS dose ≥4 times than the dose at baseline or,
   Baseline ICS dose is defined as the dosage the subject received during the 30-day run-in period,
a decrease in peak flow of 30% or more (compared to baseline) on 2 consecutive days of treatment or,
   Baseline peak flow will be determined by the average of measurements in the 7 days preceding Visit 2,
an asthma exacerbation requiring the use of systemic corticosteroids (tablets, suspension, or injection) for at least 3 days or,
a hospitalization or emergency room visit because of an asthma exacerbation.

Time to event will be measured in days using the first day of the event above to denote the day of the overall asthma related event occurrence.

Patients meeting any of the above criteria for an asthma related event will be permitted to restart background (pre-study) therapy. If background therapy is reinitiated, the patient must be withdrawn from the study.

### Forced Expiratory Volume in 1 Second (FEV₁)

The FEV₁ is the volume of air that can be forcibly exhaled from the lungs in the first second, measured in liters by a spirometer and reported as a percent of expected volume.

### Fractional Exhaled Nitric Oxide (FeNO)

A FeNO test measures the levels of nitric oxide during exhalation. A FeNO test will be done by breathing into a tube attached to a hand-held monitor.

### Peak Flow Meter

A peak flow meter measures how well lungs are able to expel air. Subjects will measure peak flow daily on a device provided by the sponsor and record the results in their diary.

### Asthma Control Questionnaire (ACQ)

This is a simple questionnaire to measure the adequacy of asthma control and change in asthma control. ACQ has a multidimensional construct assessing symptoms (5 items, self-administered), rescue bronchodilator use (1 item, self-administered), and FEV₁(1 item, completed by study staff). Scores range between 0 (totally controlled) and 6 (severely uncontrolled). (Juniper E F, Svensson K, Mörk AC, Ståhl E. Respir. Med. 2005; 99(5): 553-558).

### Standardized Asthma Quality of Life Questionnaire for 12 Years and Older (AQLQ(S)+12)

The AQLQ(S)+12 is a modified version of the standardized AQLQ, which was developed to measure functional impairments experienced by adults aged ≥17 years. The AQLQ(S)+12 is valid for patients aged 12 to 70 years and includes 32 questions in 4 domains (symptoms, activity limitation, emotional function, and environmental stimuli). (Juniper et al. Thorax. 1992; 47(2): 76-83.; Wyrwich et al. Qual Life Res. 2011; 20(6): 903-912).

Subjects will be asked to recall their experiences during the previous 2 weeks and score each of the questions on a 7-point scale, where 7=not at all limited and 1=totally limited. The overall score of the AQLQ+12 will be derived as the average of the 32 questions; thus, the total score ranges from 1 (indicates “total impairment”) to 7 (indicates “no impairment”).

### Asthma Symptom Diary

The Asthma Symptom Diary is a 6-item daily measure of asthma symptom severity that assesses three core categories of asthma symptoms: breathing symptoms (difficulty breathing; wheezing; shortness of breath), chest symptoms (chest tightness; chest pain), and cough. The Asthma Symptom Diary is intended for twice daily completion and comprises a morning diary (for completion upon waking and referring to asthma symptoms during the nighttime) and an evening diary (for completion before going to bed and referring to asthma symptoms during the day). Subjects are required to rate the 6 symptoms at their worst during the respective timeframes using an 11-point numeric rating scale ranging from 0 (‘None’) to 10 (‘As bad as you can imagine’). (United States Food and Drug Administration. Clinical Outcome Assessments (COA) Qualification Program DDT COA #000006: Asthma Daytime Symptom Diary (ADSD) and Asthma Nighttime Symptom Diary (ANSD), available at https://www.fda.gov/drugs/clinical-outcome-assessment-coa-qualification-program/ddt-coa-000006-asthma-daily-symptom-diary-adsd).

### European Quality of Life-5 Dimension 5 Level Questionnaire (EQ-5D-5L)

The EQ-5D-5L consists of the EQ-5D descriptive system and the EQ visual analogue scale (EQ VAS). The descriptive system comprises five dimensions: mobility, self-care, usual activities, pain/discomfort, and anxiety/depression. Each dimension has 5 levels: no problems, slight problems, moderate problems, severe problems, and extreme problems. The EQ VAS records the subject's self-rated health on a vertical VAS, where the endpoints are labelled ‘The best health you can imagine’ and ‘The worst health you can imagine’ (EuroQol, Group. EQ-5D-5L: About. 2017, available at https://euroqol.org/eq-5d-instruments/eq-5d-51-about/EuroQol, 2017).

### Patient Global Impression of Change/Severity

The Patient Global Impression of Change or Severity (PGI-C, PGI-S) scale is a global index that can be used to rate the severity of a specific condition (a single-state scale) from the patient's perspective. The PGI-C is a single question scale asking the patient to rate the overall status of their specific condition on a 7 point scale. The PGI-S is a single question scale asking the patient to rate current state of their specific condition on a 7 point scale.

### Clinician Global Impression of Improvement/Severity

The Clinician Global Impression of Improvement or Severity (CGI-I, CGI-S) is a global index that can be used to rate the severity of a specific condition (a single-state scale) from the clinician's perspective. The CGI-I is a single question scale asking clinician to rate the overall status of the patient's specific condition on a 7 point scale since the beginning of the research study. The CGI-S is a single question scale asking the clinician to rate current state of the patient's specific condition on a 7 point scale.

### Short-Acting Beta Agonist Use

The number of times a short-acting beta agonist (number of inhalations/puffs) is used will be assessed by reviewing the diary maintained by the subject.

### Safety

Safety and tolerability assessments will include the frequency and severity of AEs as well as the evaluation of changes in clinical laboratory values, vital signs, and physical examination findings.

### Clinical Laboratory Tests to be Performed

Samples for the following clinical laboratory tests will be collected at the time points specified in the schedule of assessments (Table 1).

Hematology tests include hemoglobin, hematocrit, red blood cell count, red blood cell indices, mean corpuscular hemoglobin, mean corpuscular hemoglobin concentration, mean corpuscular volume, platelet count (or estimate), and white blood cell count including differential.

Serum chemistry tests include sodium, potassium, chloride, bicarbonate, glucose, blood urea nitrogen, creatine, creatinine phosphokinase, alanine aminotransferase, aspartate aminotransferase, gamma-glutamyl transferase, alkaline phosphatase, total bilirubin, calcium, magnesium, phosphorous, lactate dehydrogenase, uric acid, total protein, albumin, C-reactive protein, total cholesterol, high density lipoprotein, LDL, and triglycerides.

A serum/urine pregnancy test will be administered for females of childbearing potential.

Urinalysis tests include pH, specific gravity, dipstick determinations of protein, blood, glucose, and ketones.

Other tests include anti-drug antibody (ADA), serum: soluble LIGHT, inflammatory proteins, immunophenotyping, sputum: soluble LIGHT, and Plasma Antibody A Pharmacokinetics.

Fasting is not required for any study-specific lab sample.

Laboratory specimens will be collected and analyzed at the laboratories specified in the study Laboratory Manual(s) or guidelines.

### Sampled Blood Volume

The sampled blood volume for this study is shown in Table 2.

TABLE 2
Sampled Blood Volume per Subject

| | Sample | | |
|---|---|---|---|
| | Volume | Number of | Total Volume |
| Assessment | (mL) | samples | (mL) |
| Chemistry | 3.5 | 8 | 28.0 |
| Hematology | 2.0 | 8 | 16.0 |
| ADA | 4.5 | 7 | 31.5 |
| Serum LIGHT | 3.5 | 7 | 24.5 |
| Inflammatory proteins | 3.5 | 5 | 17.5 |
| Immunophenotyping of circulating | 10.0 | 3 | 30.0 |
| immune cells | | | |
| Plasma Antibody A | 4.5 | 7 | 31.5 |
| Total (up to) | 31.5 | 45 | 179 |

ADA = anti-drug antibody; LIGHT = Lymphotoxin-like, exhibits Inducible expression, and competes with Herpes Virus Glycoprotein D for Herpesvirus Entry Mediator, a receptor expressed by T lymphocytes.

### Evaluation of Laboratory Values

The normal ranges of values for the central laboratory assessments in this study will be provided to each site as part of the lab manual. For assessments performed locally, the normal ranges of values for the local laboratory will be provided to the sponsor's designee. They will be regarded as the reference ranges on which decisions will be made for the specific site.

If a laboratory value is out of the reference range, it is not necessarily clinically relevant. The investigator must evaluate the out-of-range values and record his/her assessment of the clinical relevance in the subject's source documentation.

All laboratory values which, in the investigator's opinion, show clinically relevant or pathological changes during or after termination of the treatment are to be discussed with the medical monitor, as necessary, and reported as AEs and followed.

All measurements described in this section are recognized standard methods.

### Clinical Examination: Vital Signs

Vital signs, including systolic and diastolic blood pressure, temperature, pulse, respiration rate, height, and bodyweight, will be collected as shown in the schedule of assessments (see Table 1).

Pre-dose vital signs should be taken within 60 minutes before dosing. Post-dose vital signs should be taken at least 60 minutes after dosing, prior to discharge. Additional blood pressure and pulse measurements may be performed, as determined by the investigator, to ensure appropriate monitoring of subject safety and accurate recording of vital sign measurements. Any changes from baseline deemed clinically significant by the investigator are to be recorded as AEs.

### Clinical Examination: Electrocardiogram

A standard 12 lead ECG will be performed after the subject has been supine for approximately 5 minutes, at time points shown in the schedule of assessments (see Table 1). All ECG recordings will be identified with the subject number, date, and time of the recording and a copy will be included with the subject's source documentation.

All ECG values which, in the investigator's opinion, show clinically relevant or pathological changes during or after termination of the treatment are to be discussed with the medical monitor and reported as AEs and followed.

### Clinical Examination: Physical Examination

A complete physical examination, including measurements of height and weight, will be conducted by a qualified licensed physician, physician's assistant, or a nurse practitioner at Visit 1 through Visit 9/ET (see Table 1). Any clinically significant physical examination findings are to be reported as AEs and followed. Height will be measured only at Visit 1.

### Clinical Examination: Adverse Events

The investigator is responsible for the detection and documentation of events meeting the criteria and definition of an AE or SAE described below. At each visit, the subject will be allowed time to spontaneously report any issues since the last visit or evaluation.

Any clinically relevant observations made during each visit will also be considered AEs. AEs will be collected from the time of informed consent through the last study visit.

### Immunogenicity Analyses

Blood samples for ADA analysis will be collected at time points shown in the schedule of assessments (see Table 1). The Visit 2 sample is to be collected prior to dosing. Additionally, an ADA sample will be collected if an immunologically related AE is reported. Samples for ADA will be processed according to the methods and directions set forward in the laboratory manual(s) and guidance(s). ADA sample analysis will be performed by a laboratory defined in the laboratory manual(s) and guidance(s), according to their standard operating procedures (SOPs) using a validated method. Assay and analysis details will be described in the method validation and bioanalytical information.

### Pharmacokinetic, Pharmacodynamic and Biomarker Assessments

Blood samples will be collected for assessment of PK, PD activity and mechanism of action of Antibody A in NEA.

Blood samples will be used to isolate serum, which will be subjected to analysis of soluble LIGHT levels in circulation using a relevant immunoassay for PD activity measures. Additionally, for selected subset of sites or subjects, sputum samples will be analyzed for soluble LIGHT levels and gene expression. Biomarkers associated with inflammation such as (including but not limited to) IL-6, IFN-γ, and TNF-α may be evaluated using relevant immunoassays. Scrum may be also used for novel biomarkers analyses as the rationale evolves.

Blood samples collected for PBMC isolation may be used for comprehensive immunophenotyping of immune cell subsets in circulation by flow cytometry or CyTOF to study the effects of AVTX-002 on these cell types. Furthermore, transcriptomic analysis may be performed in PBMC as needed.

All these exploratory biomarkers as listed in Table 1 will be performed at the laboratories specified in the laboratory manual(s) and/or guidance(s).

### Example 1.5—Adverse Events

An AE is defined as any untoward medical occurrence in a patient or clinical investigation subject administered a pharmaceutical product that does not necessarily have a causal relationship with the product. An AE can therefore be any unfavorable and unintended sign (including a new, clinically important abnormal laboratory finding), symptom, or disease, temporally associated with the product, whether related to the product. An AE will be considered treatment-emergent if it occurs after the first dose of investigational product and within 28 days of a subject's last dose of investigational product. Additionally, an AE that occurred prior to dosing with study medication and increased in severity after start of dosing and within 28 days of a subject's last dose of investigational product will also be considered treatment emergent.

All AEs are collected from the time of the informed consent is signed until the end of study (Day 98/ET). This includes events occurring regardless of whether investigational product is administered. Note: Clinically significant observations noted during screening procedures (labs, physical examination, vital signs, etc.) should be entered as medical history. Only if the clinically significant observation is clearly related to the performance of a screening procedure should it be entered as an AE (e.g., a hematoma because of drawing blood for screening labs). Where possible, a diagnosis rather than a list of symptoms should be recorded. If a diagnosis has not been made, then each symptom should be listed individually.

All AEs must be followed to closure, regardless of whether the subject is still participating in the study. Closure indicates that an outcome is reached or stabilization achieved (i.e. the investigator does not expect any further improvement or worsening of the event). When appropriate, medical tests and examinations are performed so that resolution of an event(s) can be documented.

Lack of effect, including worsening of symptoms, disease progression, or lack of improvement, should not be recorded as an AE unless it meets the definition of the criteria for an SAE.

An AE that changes in severity over time should be recorded in the eCRF once at the highest severity with two exceptions. The first exception is worsening of non-NEA-related pre-treatment events after initiation of investigational product must be recorded as new AEs. For example, if the subject experiences mild, intermittent headaches prior to dosing with investigational product; however, the headache intensity increases to moderate after the first dose of investigational product, a new AE of moderate intermittent headaches is to be recorded in the source documents and eCRF. The second exception is an AE which begins as a non-serious event, which later meets the definition of an SAE, should be entered once for the non-serious portion of the AE, and then be re-recorded as a new event with the start date the day it became serious.

### Severity of Adverse Events

The medical assessment of clinical severity of an AE will be determined using the definitions outlined in Common Terminology Criteria for Adverse Events (CTCAE), Version 5.0 (Published Nov. 27, 2017, by the US Department of Health and Human Services, National Institutes of Health, National Cancer Institute). Grade 1 is defined as Mild; asymptomatic or mild symptoms; or clinical or diagnostic observations only; or intervention not indicated. Grade 2 is defined as Moderate; or minimal, local or non-invasive intervention indicated; or limiting age-appropriate instrumental activities of daily living (ADL). Grade 3 is defined as Severe or medically significant but not immediately life-threatening; or hospitalization or prolongation of hospitalization indicated; or disabling; or limiting self-care ADL. Grade 4 is defined as Life-threatening consequences; or urgent intervention indicated. Grade 5 is defined as Death related to AE.

The above grading guidelines should be used whenever possible. For AEs that cannot be graded using CTCAE, the severity should be graded using mild (Grade 1), moderate (Grade 2), severe (Grade 3), life threatening (Grade 4), and fatal (Grade 5).

Please refer to the above-referenced CTCAE document for full description of CTCAE terms and instrumental and self-care ADLs. It is important to distinguish between severe AEs and SAEs. Severity is a classification of intensity whereas an SAE is an AE that meets serious criteria.

### Relationship Categorization

A physician investigator must make the assessment of relationship to investigational product for each AE. The investigator should decide whether, in his or her medical judgment, there is a reasonable possibility that the event may have been caused by the investigational product. If there is no valid reason for suggesting a relationship, then the AE should be classified as “not related”. Otherwise, the AE should be categorized per the guidelines below. The causality assessment must be documented in the source document and the eCRF (Table 3).

TABLE 3
Assessment of Relationship to Investigational Product

| Relationship | Description |
|---|---|
| Not Related | Exposure to investigational product has not occurred. |
| | OR |
| | The administration of investigational product and the |
| | occurrence of the AE are not reasonably related in time |
| | OR |
| | The AE is considered likely to be related to an etiology |
| | other than the use of the investigational product, that |
| | is, there are no facts/evidence or arguments to suggest |
| | a causal relationship to the investigational product. |
| Possibly Related | The administration of the investigational product and |
| | the occurrence of the AE are reasonably related in time. |
| | AND |
| | The AE could not be explained equally well by factors or |
| | causes other than exposure to investigational product |
| Probably Related | The administration of investigational product and the |
| | occurrence of the AE are reasonably related in time. |
| | AND |
| | The AE is more likely explained by exposure to |
| | investigational product than by other factors or causes. |

AE = adverse event.

### Outcome at the Time of Last Observation

The outcome at the last observation will be classified as: recovered/resolved; recovered/resolved with sequelae; recovering/resolving; not recovered/not resolved; fatal; or unknown.

### Reporting of Serious Adverse Events

Initial and follow-up SAE reports must be completed by the investigator or designee and sent to the CRO within 24 hours of the first awareness of an SAE. The investigator or designee must complete, sign and date the appropriate SAE form and verify the accuracy of the information against corresponding source documents. This information is to be sent to the CRO Pharmacovigilance Department.

### Serious Adverse Event Definition

An SAE is any untoward medical occurrence, whether considered to be related to investigational product or not, that at any dose: results in death; is life-threatening; requires inpatient hospitalization or prolongation of existing hospitalization; results in persistent or significant disability/incapacity; is a congenital anomaly; or is an important medical event.

Note that the term “life-threatening” in the definition of “serious” refers to an event in which the subject was at risk of death at the time of the event; it does not refer to an event which hypothetically might have caused death if it were more severe.

Note that inpatient hospitalization is defined as 24 hours in a hospital or an overnight stay. An elective hospital admission to treat a condition present before exposure to the test drug, or a hospital admission for a diagnostic evaluation of an AE, does not qualify the condition or event as an SAE. Further, an overnight stay in the hospital that is only due to transportation, organization, or accommodation problems and without medical background does not need to be considered an SAE.

Note that a congenital anomaly in an infant born to a mother who was exposed to the investigational product during pregnancy is an SAE. However, a newly diagnosed pregnancy in a subject that has received an investigational product is not considered an SAE unless it is suspected that the investigational product interacted with a contraceptive method and led to the pregnancy.

Note that medical and scientific judgment should be exercised in deciding whether it is appropriate to consider other situations serious, such as important medical events that may not be immediately life-threatening or result in death or hospitalization but may jeopardize the subject or may require intervention to prevent one of the other outcomes listed in the definition above. Examples of such events are intensive treatment in an emergency room or at home for allergic bronchospasm, blood dyscrasias or convulsions that do not result in hospitalization, or development of drug dependency or drug abuse.

### Serious Adverse Event Collection Time Frame

All SAEs, regardless of the relationship to study, are collected from the time the subject signs the informed consent until the End of Study/Early Termination Visit. The investigator or designee must report all SAEs promptly to CRO within 24 hours of first becoming aware of the event.

Any SAE(s), regardless of relationship to study, which occurred during the study but is not discovered by the site until after the study has been completed must be reported to CRO within 24 hours of the first awareness of the event.

### Serious Adverse Event Onset and Resolution Dates

The onset date of the SAE is defined as the date the event meets serious criteria. The resolution date is the date an outcome is reached or stabilization is achieved (i.e. the investigator does not expect any further improvement or worsening of the event).

Any signs or symptoms experienced by the subject after signing the informed consent form and assent form (if applicable) or leading up to the onset date of the SAE or following the resolution date of the SAE must be recorded as an AE.

### Fatal Outcome

Fatal should only be selected as an outcome when the AE results in death. If more than 1 AE is possibly related to the subject's death, the outcome of death should be indicated for each such AE.

Any AE that results in the subject's death must have fatal checked as an outcome with the date of death recorded as the resolution date. AEs resulting in death must be reported within 24 hours as a SAE, if not already reported as such. In the event of a subject's death, data should be collected on whether the death occurred after the withdrawal of care and, if so, the reason for the withdrawal of care.

For other AEs, ongoing at the time of death that did not contribute to the subject's death, the outcome should be considered not resolved, without a resolution date recorded.

### Pregnancy

All females of childbearing potential, and males with female partners of childbearing potential, who participate in the study should be counseled on the need to utilize a highly effective method of birth control throughout the study and for 28 days following the last dose of study drug, and on the importance of avoiding pregnancy during study participation. A highly effective method of birth control is defined as one that results in a low failure rate (i.e., <1% per year) when used consistently and correctly, such as oral/injectable/inserted/implanted/transdermal contraceptives, condom with diaphragm, condom with spermicide, diaphragm with spermicide, intrauterine hormone-releasing system or IUD, or sexual abstinence. Contraception is not required where at least 6 weeks have passed since sterilization, defined as females having undergone one of the following surgeries: hysterectomy, bilateral tubal ligation or occlusion, bilateral oophorectomy, or bilateral salpingectomy; and males who are vasectomized. Contraception is not required where females are postmenopausal (12 consecutive months of spontaneous amenorrhea and age ≥51 years).

Females and males with female partners should be instructed to contact the investigator or study staff immediately if pregnancy occurs or is suspected.

Pregnancy testing will be conducted on every female as per the schedule of assessments (Table 1). A female who is found to be pregnant at screening will be excluded from the study and considered to be a screening failure. A female who is found to be pregnant after receiving investigational product is required to be discontinued from the study and the end of study visit assessments performed as soon as possible after learning of the pregnancy.

The investigator must report the pregnancy of any female (study participant or female partner of male study participant) who becomes pregnant during investigational product treatment or within 28 days of discontinuing the investigational product (permission must be obtained from the pregnant female partner of a male subject to follow the pregnancy to conclusion and report the results). The pregnancy must be reported within 24 hours of learning of the pregnancy to the CRO using the Pregnancy Data Collection Form via the same email address as for SAE reporting. The investigator should contact the designated individual(s) who receive pregnancy notification and record information related to the pregnancy on the Pregnancy Form/other designated form provided by the sponsor or its designee.

The investigator is also responsible for following the pregnancy until delivery or termination. These findings must be reported on the Pregnancy Data Collection Form and forwarded to the designated individual(s). The event meets the SAE criterion only if it results in a spontaneous abortion or a congenital anomaly.

### Reporting to Regulatory Agency, Institutional Review Board/Ethics Committee and Site

The Sponsor or its designee is responsible for notifying the relevant regulatory authorities and if applicable, US central institutional review board (IRB) of related, unexpected SAEs.

In addition, the Sponsor and the CRO are responsible for notifying active sites of all related, unexpected SAEs occurring during all interventional studies across the development program.

The investigator is responsible for notifying the local IRB, local ethics committee, or the relevant local regulatory authority of all SAEs that occur at his/her site, as required.

### Example 1.8—Statistics

The sample size in this study will be approximately 80 with 40 subjects per treatment arm, randomized in a 1:1 ratio.

The sample size estimate was based on the estimated proportions of patients in the two treatment groups expected to have an asthma related event as defined by the primary endpoint. In a study with another product, rates of 6% in the active group and 44% in the placebo group were observed. (Wenzel et al., N Engl J Med, June 2013; 368(26): 2455-66). If these estimates were used as is, then a study with 40 subjects per group would have over 95% power. Considering likely variations from the cited study, estimates of sample were assessed assuming a Type I error of 5% and the proportion of subjects with an asthma related event ranging from 30% to 50% in the placebo group and 5% to 8% in the active group. In the 20 such scenarios examined, only in the cases in which the difference between treatments gets below 23% does the power fall slightly below 80%. In the vast majority of cases examined the power is greater than 90%.

A sample size re-estimation may be performed at a point in time when an adequate number of subjects has been enrolled to provide meaningful data. Details will be provided in the statistical analysis plan.

The study will have the following populations of interest: the Randomized Analysis Set; the Safety Analysis Set; and the Full Analysis Set. The Randomized Analysis Set will include all subjects who are randomized in the study. Subjects will be categorized according to their randomized treatment group. The Randomized Analysis Set will be used for all disposition, protocol deviations, and demographic and other baseline characteristics analyses. The Safety Analysis Set will include all subjects who are randomized in the study and receive at least one dose of investigational product. Subjects will be categorized according to their actual treatment group. The Safety Analysis Set will be used for all exposure and safety analyses and immunogenicity analysis. The Full Analysis Set will include all subjects who receive at least one dose of investigational product and have a baseline and at least one post-baseline efficacy assessment. Subjects will be categorized according to their randomized treatment group. The Full Analysis Set will be used for all efficacy and pharmacodynamic analyses.

This section presents a summary of the planned statistical analyses. Additional details regarding data handling, analytical methods, and presentation of results will be described in the Statistical Analysis Plan (SAP) for this study. The SAP will be finalized prior to database lock.

All efficacy, safety, PD, and ADA variables will be summarized using descriptive statistics. Descriptive statistics for continuous data will include number of subjects (n), mean, standard deviation, median, minimum, and maximum. Summaries of change from baseline variables will include only subjects who have both a baseline value and corresponding value at the timepoint of interest. Descriptive statistics for categorical data will include frequency and percentage. Listings will be provided for all collected study data.

The study medication and study disposition of all subjects randomized in this study will be summarized by treatment group and completion/discontinuation status. Subjects who discontinue the study medication and/or study prematurely will be summarized by treatment group and reason for discontinuation. The number of subjects in each analysis set will also be summarized by treatment group.

All subject data will be reviewed for the occurrence of protocol deviations. Prior to database lock, all protocol deviations will be reviewed and classified with respect to the potential to influence experimental outcomes. Protocol deviations will be summarized by treatment group.

Demographic and other baseline characteristics will be summarized by treatment group using descriptive statistics.

All prior and concomitant medications will be coded using the WHO Drug Dictionary. Prior and concomitant medications will be summarized by treatment group using descriptive statistics.

Exposure to investigational product will be summarized by treatment group using descriptive statistics.

Safety analyses will be conducted using data from the Safety Analysis Set. Safety variables will include TEAEs, clinical laboratory values, vital signs, and ECG results. No formal inferential analyses will be conducted for any safety variables, unless otherwise noted.

Adverse event verbatim terms will be coded using the Medical Dictionary for Regulatory Activities (MedDRA). The overall incidence of subjects having at least one AE will be summarized by treatment group. The incidence of TEAEs will be summarized by treatment group, system organ class (SOC) and preferred term (PT). Each subject will be counted only once per SOC and preferred term. An AE will be considered treatment-emergent if it occurs after the first dose of investigational product and within 28 days after a subject's last dose of investigational product.

For all continuous laboratory test variables, descriptive statistics for all reported values and change from baseline values will be summarized by treatment group and visit.

For all continuous vital sign variables, descriptive statistics for all reported values and change from baseline values will be summarized by treatment group and visit.

For the primary variable, comparison of the proportions of subjects that had an asthma related event will be via a Wald Z-test (continuity corrected). Summary statistics will be provided via a 2×2 table showing the number and percentage with and without events in each treatment group. The test statistic, its associated p-value and a 95% confidence interval for the treatment difference will also be provided.

All other efficacy variables will be summarized using descriptive statistics. Descriptive statistics for continuous data will include number of subjects (n), mean, standard deviation, median, minimum, and maximum. Summaries of change from baseline variables will include only subjects who have both a baseline value and corresponding value at the timepoint of interest. Descriptive statistics for categorical data will include frequencies and percentages.

Further details regarding all statistical analysis can be found in the study Statistical Analysis Plan (SAP).

For all PK, PD and biomarker variables, descriptive statistics will be presented by treatment group and time point.

For all immunogenicity variables, descriptive statistics will be presented by treatment group and time point.

The following Table 4 provides the sequences referred to in this application.

TABLE 4
Table of Sequences

| SEQ | | |
|---|---|---|
| ID | | |
| NO | DESCRIPTION | SEQUENCE |
| 1 | Human DcR3 | MRALEGPGLS LLCLVLALPA LLPVPAVRGV |
| | amino acid | AETPTYPWRD |
| | sequence | AETGERLVCA QCPPGTFVQR PCRRDSPTTC |
| | | GPCPPRHYTQ FWNYLERCRY CNVLCGEREE |
| | | EARACHATHN RACRCRTGFF AHAGFCLEHA |
| | | SCPPGAGVIA PGTPSQNTQC QPCPPGTFSA SSSSSEQCQP |
| | | HRNCTALGLA LNVPGSSSHD TLCTSCTGFP |
| | | LSTRVPGAEE CERAVIDFVA FQDISIKRLQ RLLQALEAPE |
| | | GWGPTPRAGR AALQLKLRRR LTELLGAQDG |
| | | ALLVRLLQAL RVARMPGLER SVRERFLPVH |
| 2 | Heavy Chain | GYNWH |
| | (HC) CDR1 | |
| | antibody F19 | |
| 3 | HC CDR2 | EITHSGSTNYNPSLKS |
| | antibody F19 | |
| 4 | HC CDR3 | EIAVAGTGYYGMDV |
| | antibody F19 | |
| 5 | LC CDR1 | RASQGINSAFA |
| | antibody F19 | |
| 6 | LC CDR2 | DASSLES |
| | antibody F19 | |
| 7 | LC CDR3 | QQFNSYPLT |
| | antibody F19 | |
| 8 | Heavy chain | QVQLQQWGAG LLKPSETLSL TCAVYGGSFS |
| | antibody F19 | GYNWHWIRQP PGKGLEWIGE ITHSGSTNYN |
| | | PSLKSRVTIS VDTSKNQFSL KLSSVTAADT |
| | | AVYYCVREIA VAGTGYYGMD VWGQGTTVTV |
| | | SSASTKGPSV FPLAPCSRST SESTAALGCL VKDYFPEPVT |
| | | VSWNSGALTS GVHTFPAVLQ SSGLYSLSSV |
| | | VTVPSSSLGT KTYTCNVDHK PSNTKVDKRV |
| | | ESKYGPPCPP CPAPEFEGGP SVFLFPPKPK DTLMISRTPE |
| | | VTCVVVDVSQ EDPEVQFNWY VDGVEVHNAK |
| | | TKPREEQFNS TYRVVSVLTV LHQDWLNGKE |
| | | YKCKVSNKGL PSSIEKTISK AKGQPREPQV |
| | | YTLPPSQEEM TKNQVSLTCL VKGFYPSDIA |
| | | VEWESNGQPE NNYKTTPPVL DSDGSFFLYS |
| | | RLTVDKSRWQ EGNVFSCSVM HEALHNHYTQ |
| | | KSLSLSLG |
| 9 | Light chain | AIQLTQSPSS LSASVGDRVT ITCRASQGIN |
| | antibody F19 | SAFAWYQQKP GKAPKLLIYD ASSLESGVPS |
| | | RFSGSGSGTD FTLTISSLQP EDFATYYCQQ FNSYPLTFGG |
| | | GTKVEIKRTV AAPSVFIFPP SDEQLKSGTA SVVCLLNNFY |
| | | PREAKVQWKV DNALQSGNSQ ESVTEQDSKD |
| | | STYSLSSTLT LSKADYEKHK VYACEVTHQG |
| | | LSSPVTKSFN RGEC |
| 10 | Alternative LC | RASRGINSAFA |
| | CDR1 antibody | |
| | F19 | |
| 11 | Alternative LC | DASSLES |
| | CDR2 antibody | |
| | F19 | |
| 12 | Alternative LC | QQFNSYPLT |
| | CDR3 antibody | |
| | F19 | |
| 13 | Alternative LC | RVSQGISSYLN |
| | CDR1 antibody | |
| | F19 | |
| 14 | Alternative LC | SASNLQS |
| | CDR2 antibody | |
| | F19 | |
| 15 | Alternative LC | ARTNAPPT |
| | CDR3 antibody | |
| | F19 | |
| 16 | Alternative LC | RMSQGISSYLA |
| | CDR1 antibody | |
| | F19 | |
| 17 | Alternative LC | AASTLOS |
| | CDR2 antibody | |
| | F19 | |
| 18 | Alternative LC | QQYYSFPYT |
| | CDR3 antibody | |
| | F19 | |
| 19 | Alternative LC | RASQGVSSYLA |
| | CDR1 antibody | |
| | F19 | |
| 20 | Alternative LC | DASNRAT |
| | CDR2 antibody | |
| | F19 | |
| 21 | Alternative LC | QQRSNWHP |
| | CDR3 antibody | |
| | F19 | |
| 22 | HC CDR1 | RFNMN |
| | antibody E1 | |
| 23 | HC CDR2 | YISSSSYTIYYADSVKG |
| | antibody E1 | |
| 24 | HC CDR3 | SIAAFDY |
| | antibody E1 | |
| 25 | LC CDR1 | RASQGISSALA |
| | antibody E1 | |
| 26 | LC CDR2 | DASSLES |
| | antibody E1 | |
| 27 | LC CDR3 | QQFNSYRT |
| | antibody E1 | |
| 28 | Alternative LC | RASQSVSSSYLT |
| | CDR1 antibody | |
| | E1 | |
| 29 | Alternative LC | GASSRAT |
| | CDR2 antibody | |
| | E1 | |
| 30 | Alternative LC | QQYGSSMYT |
| | CDR3 antibody | |
| | E1 | |
| 31 | Alternative LC | RASQSVSSSYLA |
| | CDR1 antibody | |
| | E1 | |
| 32 | Alternative LC | GASNRAT |
| | CDR2 antibody | |
| | E1 | |
| 33 | Alternative LC | QQYGSSPWT |
| | CDR3 antibody | |
| | E1 | |
| 34 | HC CDR1 | NAWMS |
| | antibody E13 | |
| 35 | HC CDR2 | RIKSKIDGGTTDYAAPVKG |
| | antibody E13 | |
| 36 | HC CDR3 | AMAGAFGF |
| | antibody E13 | |
| 37 | LC CDR1 | RASQSVSSSYLA |
| | antibody E13 | |
| 38 | LC CDR2 | GASSRAT |
| | antibody E13 | |
| 39 | LC CDR3 | QQYGSSPMYT |
| | antibody E13 | |
| 40 | HC CDR1 | SGGYYWS |
| | antibody E63 | |
| 41 | HC CDR2 | YIYYSGSTNYNPSLKS |
| | antibody E63 | |
| 42 | HC CDR3 | WITMFRGVGFDP |
| | antibody E63 | |
| 43 | LC CDR1 | RASQSIGSSLH |
| | antibody E63 | |
| 44 | LC CDR2 | YASQSFS |
| | antibody E63 | |
| 45 | LC CDR3 | RQSSSLPLT |
| | antibody E63 | |
| 46 | HC CDR1 | GYYWN |
| | antibody F23 | |
| 47 | HC CDR2 | EINQYNPSLKS |
| | antibody F23 | |
| 48 | HC CDR3 | EIAIADKGYYGLDV |
| | antibody F23 | |
| 49 | LC CDR1 | RASQGISSALA |
| | antibody F23 | |
| 50 | LC CDR2 | DASSLES |
| | antibody F23 | |
| 51 | LC CDR3 | QQFNSYPLT |
| | antibody F23 | |
| 52 | HC CDR1 | SYYIH |
| 53 | HC CDR2 | PGSDITKYNEKFKG |
| 54 | HC CDR3 | GISTYSAMDF |
| 55 | LC CDR1 | KASQDVGTAVA |
| 56 | LC CDR2 | WASTRHT |
| 57 | LC CDR3 | QQYSSYPLT |
| 58 | HC variable | QVQLVQSGAE VKKPGASVKV SCKASGYTFT |
| | region | SYYIHWVRQA PGQRLEWMGW IFPGSDITKY |
| | | NEKFKGRVTI TRDTSASTAY MELSSLRSED |
| | | TAVYYCARED YGISTYSAMD FWGQGTLVTV SS |
| 59 | LC variable | DIQLTQSPSF LSASVGDRVT ITCKASQDVG |
| | region | TAVAWYQQKP GKAPKLLIYW ASTRHTGVPS |
| | | RFSGSGSGTE FTLTISSLQP EDFATYYCQQ |
| | | YSSYPLTFG QGTKVEIKR |
| 60 | HC CDR1 of | HFDIN |
| | 18E04 | |
| 61 | HC CDR2 of | WMNPDSDNTDYAQEFQG |
| | 18E04 | |
| 62 | HC CDR3 of | GGTTLDY |
| | 18E04 | |
| 63 | LC CDR1 of | SGDALPKKYAY |
| | 18E04 | |
| 64 | LC CDR2 of | EDSKRPS |
| | 18E04 | |
| 65 | LC CDR3 of | YSTDSSDNHVI |
| | 18E04 | |
| 66 | HC CDR1 of | DYYMS |
| | 98C07 | |
| 67 | HC CDR2 of | YISRSSFIYYSESVKG |
| | 98C07 | |
| 68 | HC CDR3 of | WELSPFDY |
| | 98C07 | |
| 69 | LC CDR1 of | RASQGISNYLA |
| | 98C07 | |
| 70 | LC CDR2 of | AASSLOS |
| | 98C07 | |
| 71 | LC CDR3 of | QQYNTYPFT |
| | 98C07 | |
| 72 | HC CDR1 of | YYGIS |
| | 1C02 | |
| 73 | HC CDR2 of | WISANSGNTNYAQKFQG |
| | 1C02 | |
| 74 | HC CDR3 of | GGVAVLEY |
| | 1C02 | |
| 75 | LC CDR1 of | WASQGISSYLA |
| | 1C02 | |
| 76 | LC CDR2 of | VASTLQS |
| | 1C02 | |
| 77 | LC CDR3 of | QQLKIYPLT |
| | 1C02 | |
| 78 | HC CDR1 of | DYYMN |
| | 1C06 | |
| 79 | HC CDR2 of | DISSRDNTIYYADSVKG |
| | 1C06 | |
| 80 | HC CDR3 of | ARERGFGDYFGMDV |
| | 1C06 | |
| 81 | LC CDR1 of | RASQDISSALA |
| | 1C06 | |
| 82 | LC CDR2 of | DASSLES |
| | 1C06 | |
| 83 | LC CDR3 of | QQFNTYPLT |
| | 1C06 | |
| 84 | Heavy chain | QVQLQQWGAGLLKP SETLSLTCAVYGGSFSGYNWH |
| | variable region | WIRQPPGKGLEWIGEITHSGSTNYNPSLK |
| | (VH) of antibody | SRVTISVDTSKNQFSLKLSSVTAADTAVYYCVR |
| | F19 | EIAVAGTGYYGMDVWG QGTTVTVSS |
| 85 | Light chain | AIQLTQSPSSLSASVGDRVTITCR |
| | variable region | ASQGINSAFAWYQQKPGKAPKLLI YDASSLESGVPSRFSG |
| | (VL) of antibody | SGSGTDFTLTISS |
| | F19 | LQPEDFATYYCQQFNSYPLTFGGGTKVEIK |

1.-54. (canceled)

55. A method of treating non-eosinophilic asthma (NEA), comprising administering an effective amount of an anti-LIGHT antibody to a human subject in need thereof, wherein the anti-LIGHT antibody comprises a heavy chain comprising a heavy chain variable region (VH) comprising a complementarity determining region 1 (CDR-H1) comprising the amino acid sequence of SEQ ID NO: 2, CDR-H2 comprising the amino acid sequence of SEQ ID NO: 3, CDR-H3 comprising the amino acid sequence of SEQ ID NO: 4, and a light chain comprising a light chain variable region (VL) comprising CDR-L1 comprising the amino acid sequence of SEQ ID NO: 5, CDR-L2 comprising the amino acid sequence of SEQ ID NO: 6, and CDR-L3 comprising the amino acid sequence of SEQ ID NO: 7.

56. The method of claim 55, wherein the VH comprises an amino acid sequence having at least 90% identity to SEQ ID NO: 84 and the VL comprises an amino acid sequence having at least 90% identity to SEQ ID NO: 85.

57. The method of claim 55, wherein the VH comprises the amino acid sequence of SEQ ID NO: 84 and the VL comprises the amino acid sequence of SEQ ID NO: 85.

58. The method of claim 55, wherein the heavy chain comprises an amino acid sequence having at least 95% identity to SEQ ID NO: 8 and the light chain comprises an amino acid sequence having at least 95% identity to SEQ ID NO: 9.

59. The method of claim 55, wherein the heavy chain comprises the amino acid sequence of SEQ ID NO: 8 and the light chain comprises the amino acid sequence of SEQ ID NO: 9.

60. The method of claim 55, wherein the anti-LIGHT antibody is administered at a dose of 600 mg.

61. The method of claim 60, wherein the anti-LIGHT antibody is administered every 14 days, every 21 days, every 28 days, every 35 days, every 42 days, every 49 days, every 56 days, or monthly.

62. The method of claim 55, wherein the anti-LIGHT antibody is administered every 28 days.

63. The method of claim 55, wherein the anti-LIGHT antibody is administered monthly.

64. The method of claim 55, wherein the anti-LIGHT antibody is administered at a dose of 600 mg every 28 days.

65. The method of claim 55, wherein the anti-LIGHT antibody is administered at a dose of 600 mg monthly.

66. The method of claim 64, wherein at least three doses of the anti-LIGHT antibody are administered.

67. The method of claim 64, wherein the anti-LIGHT antibody is administered subcutaneously.

68. The method of claim 55, wherein the administration of the anti-LIGHT antibody reduces serum total LIGHT in the subject.

69. The method of claim 64, wherein the subject has elevated total LIGHT levels as measured at baseline.

70. The method of claim 55, wherein administration of the anti-LIGHT antibody decreases the proportion of subjects in a population of subjects with an asthma related event, wherein the population of subjects has elevated total LIGHT levels as measured at baseline.

71. The method of claim 69, wherein administration of the anti-LIGHT antibody reduces the number of asthma related events for the subject.

72. The method of claim 69, wherein administration of the anti-LIGHT antibody increases the subject's time to an asthma related event.

73. The method of claim 72 wherein the asthma related event is:
a. ≥6 additional reliever puffs of short-acting beta agonist (SABA) compared to baseline in a 24-hour period on 2 consecutive days, wherein baseline SABA use is determined by the average use in the 7 days preceding administration of a first dose of the anti-LIGHT antibody;
b. increase in ICS dose ≥4 times than the dose at baseline, wherein baseline ICS dose is defined as the dosage the subject received during the 30 days leading up to administration of a first dose of the anti-LIGHT antibody;
c. a decrease in peak flow of 30% or more compared to baseline on 2 consecutive days of treatment, wherein baseline peak flow is determined by the average of measurements in the 7 days preceding administration of a first dose of the anti-LIGHT antibody;
d. an asthma exacerbation requiring the use of systemic corticosteroids for at least 3 days; or
e, wherein the asthma related event is a hospitalization or emergency room visit because of an asthma exacerbation.

74. A method of treating non-eosinophilic asthma (NEA), comprising administering an effective amount of an anti-LIGHT antibody to a human subject in need thereof, wherein the anti-LIGHT antibody comprises a heavy chain comprising a heavy chain variable region (VH) comprising a complementarity determining region 1 (CDR-H1) comprising the amino acid sequence of SEQ ID NO: 2, CDR-H2 comprising the amino acid sequence of SEQ ID NO: 3, CDR-H3 comprising the amino acid sequence of SEQ ID NO: 4, and a light chain comprising a light chain variable region (VL) comprising CDR-L1 comprising the amino acid sequence of SEQ ID NO: 5, CDR-L2 comprising the amino acid sequence of SEQ ID NO: 6, and CDR-L3 comprising the amino acid sequence of SEQ ID NO: 7, and wherein the method further comprises assaying LIGHT prior to administration of the anti-LIGHT antibody.

75. The method of claim 74, wherein the method further comprises assaying total LIGHT after administration of the anti-LIGHT antibody.
